(19) United States
(12) Patent Application Publication
(10) Pub. No.: US 2023/0277669 A1
(43) Pub. Date: Sep. 7, 2023

(54) USES OF UROLITHINS

(71) Applicant: Amazentis SA, Lausanne (CH)

(72) Inventors: Christopher L. Rinsch, Lausanne (CH); Anurag Singh, Lausanne (CH); Florian F. Greten, Frankfurt (DE); Dominc Denk, Frankfurt (DE)

(21) Appl. No.: 18/114,092

(22) Filed: Feb. 24, 2023

(30) Foreign Application Priority Data
Feb. 24, 2022 (GB) 2202515.9
Mar. 31, 2022 (GB) 2204679.1
Oct. 16, 2022 (GB) 2215264.9

(51) Int. Cl. A61K 39/00; A61K 45/06; A61P 35/00; C12N 5/0783; C12N 15/86

(52) U.S. Cl. CPC A61K 39/4631; A61K 39/4611; A61K 45/06; A61P 35/00; C12N 5/0636; C12N 15/86; C12N 2501/999; C12N 2740/15043

(57) ABSTRACT

Disclosed are improved methods for the treatment of diseases, disorders and conditions, such as cancer, using cell populations treated with a urolithin or urolithins, specifically urolithin-treated T-cell populations, for example, by enhancing the immune response comprising enhancement of antigen presentation and/or through enhancement of the expansion of T memory stem cells. Furthermore, disclosed are methods for overcoming or reversal of T-cell dysfunction to help treat diseases, such as cancer. More specifically, for example, disclosed are methods for overcoming of T-cell exhaustion. Also disclosed are cell populations enriched in T-memory stem cells.

### RELATED APPLICATIONS

This application claims the benefit of priority to Great Britain (UK) Patent Application Serial Nos. 2215264.9, filed Oct. 16, 2022; 2204679.1, filed Mar. 31, 2022; and 2202515.9, Feb. 24, 2022.

### BACKGROUND

The invention relates to improved methods for the treatment of disease, disorders and conditions, such as cancer using cell populations treated with a urolithin or urolithins, specifically urolithin-treated T-cell populations, for example, by enhancing the immune response comprising enhancement of antigen presentation and/or through enhancement of expansion of T memory stem cells. Furthermore, the invention relates to the overcoming or reversal of T-cell dysfunction to help treat diseases, such as cancer. More specifically, for example, the invention relates to the overcoming of T-cell exhaustion. The invention also relates to the use of urolithins to overcome/reverse such T-cell dysfunction. The invention further relates to cell populations enriched in T-memory stem cells.

Urolithins, for example, urolithin A, are natural metabolite of ellagitannins (ETs), of which pomegranates present a main source (Espín et al (2013) Evidence-based complementary and alternative medicine: eCAM 2013, 270418). Metabolism into urolithins, for example, urolithin A, depends on the appropriate gut microbiome that undergoes alteration with age, limiting production of UA from ETs in less than half of the aging population (Cortés-Martín et al (2018) Food & function 9, 4100-4106). Concentrated forms of urolithin A demonstrated that dietary supplementation induces mitophagy in vivo, favourably affecting the progression of aging-related diseases (reviewed in D′Amico et al (2021) Trends in molecular medicine 27, 687-699.). Rodents fed with a urolithin A-high diet display superior muscle function and recovery of muscle function in mice with Duchenne muscular dystrophy, whereas in C.elegans, Urolithin A confers higher mitochondrial content and prolonged lifespan (Luan et al (2021) Science translational medicine 13; Ryu et al (2016) Nature medicine, 879-888). Moreover, urolithin A has immunomodulatory effects in monocytic cells attenuating inflammation in various tissues (Toney et al (2021) Biomedicines 9(2) 192) and an impact on adaptive immunity by expanding FoxP3+ T-regulatory cells (Ghosh et al(2021) The Journal of Immunology 206, 113.03) or blocking Th₁/Th₁₇ cell infiltration in a model of experimental autoimmune encephalomyelitis (EAE) (Shen et al (2021) eBioMedicine 64, 103227). In humans, commercially-available supplements containing urolithin A have been deemed safe with a favourable bioavailability profile (Andreux et al (2019) Nature metabolism 1,595-603).

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described. International Patent Publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell.

Human tumours progress despite the presence of tumour associated antigen (TAA)-specific T cells, since tumours are able to evade the body’s immune system. Many different molecular and cellular mechanisms contribute to the failure of T cells to eradicate the tumour. These include immune suppressive networks that impair ongoing T cell function and enable tumour escape. Recent studies have started to reveal the nature of effector T cells in the tumour microenvironment. Targeting T cell dysfunctional mechanisms and introducing/promoting T cell stemness are promising approaches to treat patients with cancer. Surprisingly, we have found that urolithins are useful in overcoming T-cell dysfunctions and are useful to enriching T memory stem cells in cell populations.

### SUMMARY

Disclosed are improved methods for the treatment of diseases, disorders and conditions, such as cancer, using cell populations treated with a urolithin or urolithins, specifically urolithin-treated T-cell populations, for example, by enhancing the immune response comprising enhancement of antigen presentation and/or through enhancement of the expansion of T memory stem cells. Furthermore, the invention relates to the overcoming or reversal of T-cell dysfunction to help treat diseases, such as cancer. More specifically, for example, the invention relates to the overcoming of T-cell exhaustion. The invention further relates to cell populations enriched in T-memory stem cells.

### BRIEF DESCRIPTION OF THE FIGURES

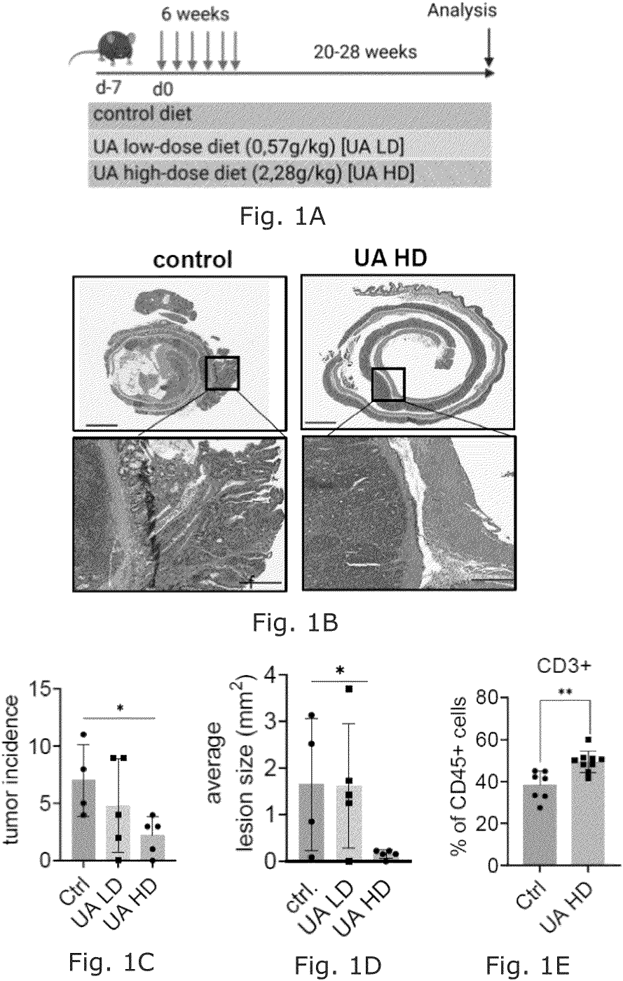

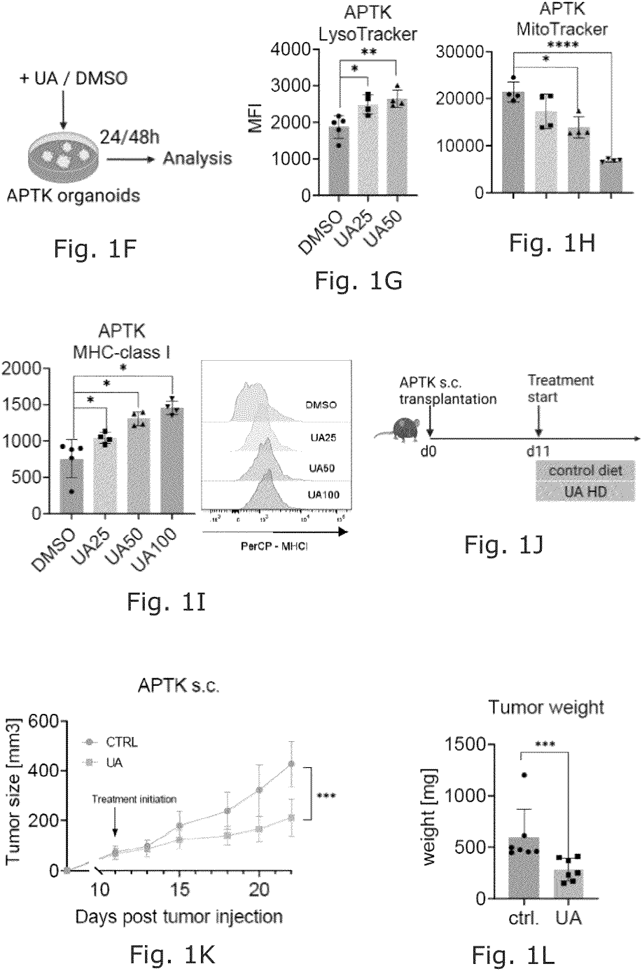

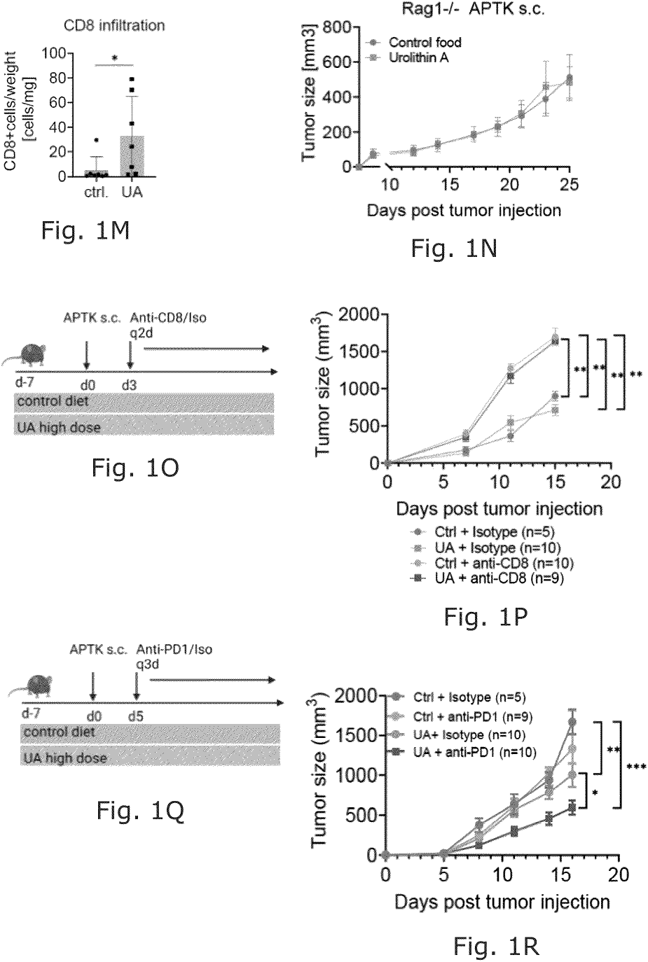

Oral UA confers anti-tumor effects in murine colorectal cancer in a T-cell dependent manner. (FIG. 1A) Schematic representation of AOM model and different UA containing diets. (FIG. 1B) Representative images of swiss-roll sections from AOM-induced colon tumors. Scale bars are 2 mm (upper row) and 300 µm (lower row), respectively. (FIG. 1C) Tumor incidence of AOM-induced tumors receiving different UA-containing diets; n=4/5/5. Data are mean ±SD, depicting one of two independent experiments. *p < 0.05 by two-sided t-test. (FIG. 1D) Average lesion size of AOM-induced tumors receiving different UA-containing diets; n=4/5/5. Data are mean ±SD, depicting one of two independent experiments. *p < 0.05 by two-sided t-test. (FIG. 1E) Relative number of CD45⁺CD3⁺ T cells in colons of AOM-treated mice at week 24 (n>7, pooled from two independent experiments). Data are mean ± SD, **p < 0.01 by two-sided t-test. (FIG. 1F) Organoid treatment scheme. APTK-organoids were incubated in the presence of UA or DMSO for the indicated time points, prior to flow cytometric analysis. (FIG. 1G) Quantification of lysosome formation as assessed by lysotracker MFI via flow cytometry after 24 h incubation at the presence of various UA concentrations (n=5/4/4). Depicts one out of two indepdent experiments. Data are mean ± SD, *p < 0.05; **p < 0.01 as established via two-sided t-test. (FIG. 1H) Mitotracker signal of APTK organoids incubated for 24 h at the presence of UA in vitro (n=4/4/4) and antigen presentation via MHC-I molecules (I) after 48 h of treatment (n=4/4/4). Results from one out of two independent experiments are shown. Data are mean ± SD, statistical analysis was performed via two-sided t-test, *p < 0.05; **p < 0.01, ****p < 0.0001. (FIG. 1I) Upregulation of MHC-I by UA. (FIG. 1J) Experimental setup of oral UA administration in mice with established APTK-s.c. tumors. Treatment diet was initiated eleven days after tumor injection and maintained until the end of the experiment. (FIG. 1K) Growth curve of mice subcutaneously transplanted with APTK tumors, receiving either UA-containing or control food (n=7 per group). Data are mean ± SD, ***p <0.001 by two-sided t-test. One out of two independent experiments are shown. (FIG. 1L) End point tumor weight, (FIG. 1M) CD8+ T cell infiltration in APTK-s.c. tumors assessed by flow cytometry, normalising total number of CD8⁺ T cells to tumor weight (n=7 per group). Data are mean ± SD, statistical analysis was performed via Mann-Whitney test, *p < 0.05 and ***p <0.001. (FIG. 1N) Size of subcutaneous APTK tumors in Rag1^{-/-} mice receiving UA-containing or control food (n=7 per group). Treatment was initiated as depicted in FIG. 1J. Data are mean +-SD, depicting one out of two independent experiments. (FIG. 1O) CD8⁺ T cell depleting or isotype control antibodies were applied every two days starting three days after s.c. injection of APTK organoids in C57BL/6 mice. (FIG. 1P) Effect of CD8⁺ T cell depletion on size of subcutaneous APTK tumors in C57BL/6 mice receiving UA-containing or control food. Data are mean ± SEM, depicting pooled data from two experiments (**p < 0.01 by two-sided t-test). (FIG. 1Q) a-PD1 or isotype antibodies were injected every three days starting five days after s.c. injection of APTK organoids in C57BL/6 mice. (FIG. 1R) Effect of a-PD1 treatment on size of subcutaneous APTK tumors in C57BL/6 mice receiving UA-containing or control food; (isotype n=5; control food+ a-PD1 n=9; other groups n=10; number shown in figure). Data shown represents pooled data from two experiments with similar results. Data are mean ± SEM (*p < 0.05; **p < 0.01 by Mann-Whitney test)

UA promotes T_{SCM} differentiation.

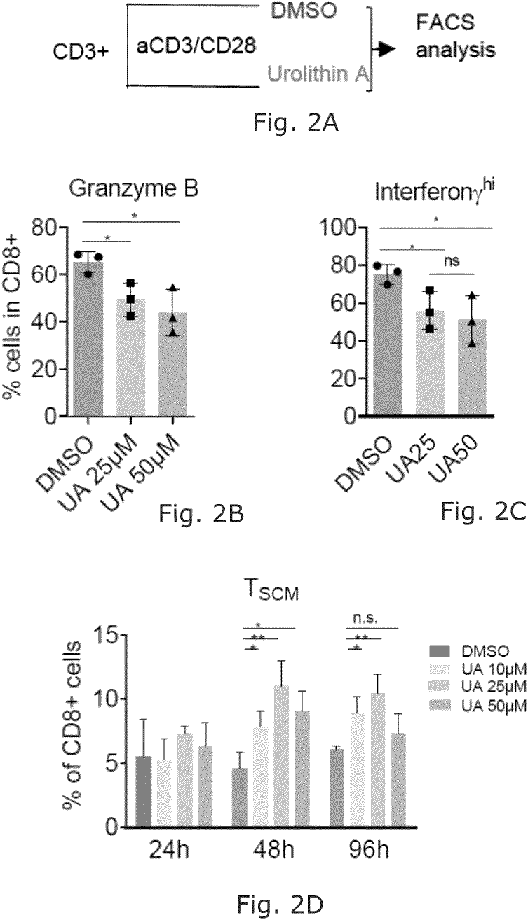

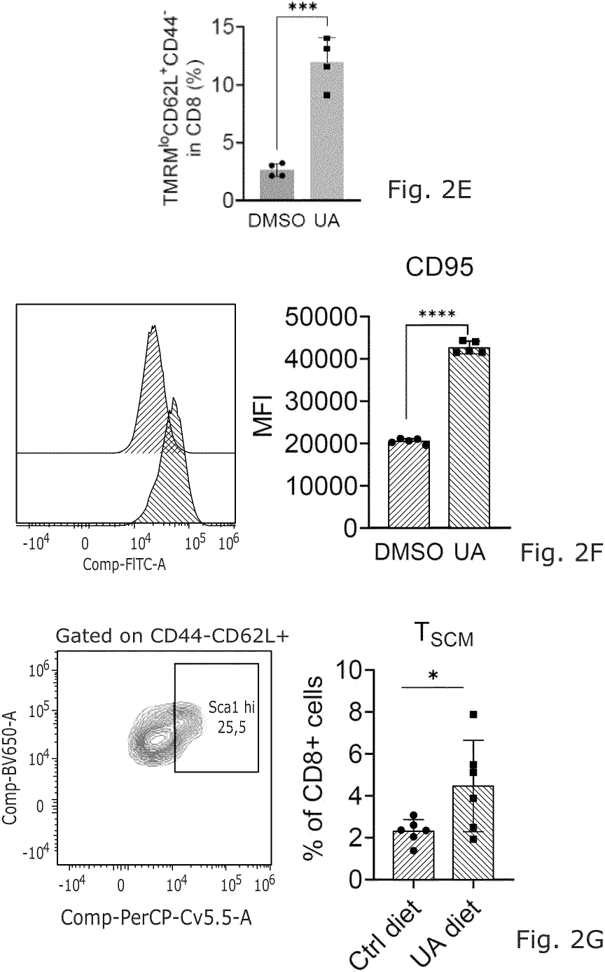

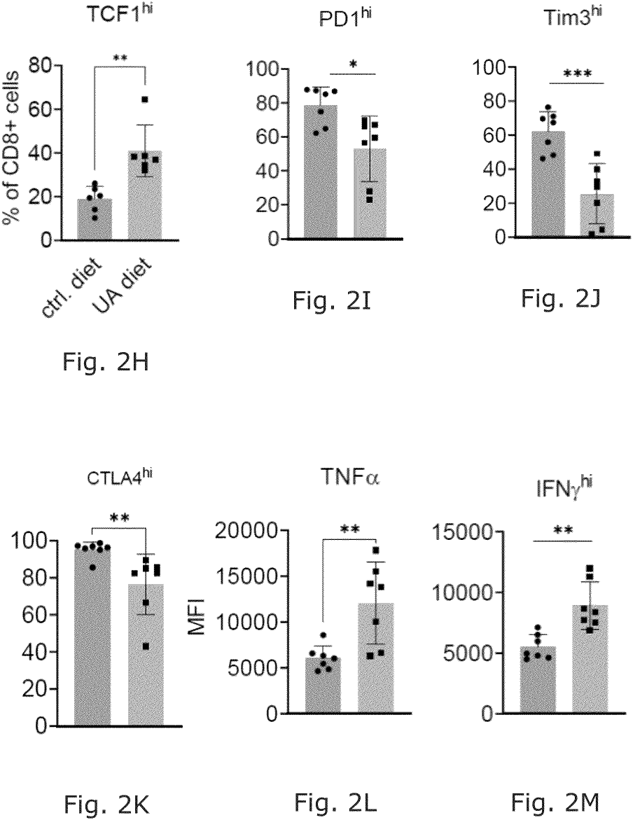

(FIG. 2A) Scheme of CD3+ T-cell activation and analysis for various timepoints. (FIG. 2B) Reduced effector molecule expression on in vitro treated cells (n=3 pooled from three independent experiments). Isolated T-cells were stimulated for 48 h at the presence of UA or DMSO in the indicated doses. Data are mean ± SD, *p < 0.05 by two-sided t-test. (FIG. 2C) Reduced effector molecule expression on in vitro treated cells (n=3 pooled from three independent experiments). Isolated T-cells were stimulated for 48 h at the presence of UA or DMSO in the indicated doses. Data are mean ± SD, *p < 0.05 by two-sided t-test. (FIG. 2D) Quantification of T memory stem cells (T_{SCM:} CD44⁻CD62L⁺Sca1^{hi}) after 24 h, 48 h and 96 h post stimulation with aCD3/aCD28 stimulation beads as indicated in (A) (n=4 pooled from four independent experiments). Data are mean ± SD, statistical analysis was performed via two-sided t-test (*p < 0.05; **p < 0.01; n.s. not significant). (FIG. 2E) Mitochondrial membrane potential within naïve CD62L⁺CD44⁻CD8⁺ T cells, with TMRM^{lo} representing cells with low mitochondrial membrane potential. Cells were stimulated for 48 h as depicted in (A). Data are mean ± SD, n=4; ***p <0.001 by two-sided t-test. Experiment was repeated twice in independent experiments. (FIG. 2F) Representative histogram and quantification of memory marker CD95 expression in CD62L⁺CD44⁻CD8⁺ cells, 48 hours after in vitro stimulation. Data are mean ± SD; n=5; ****p < 0.0001 by two-sided t-test. Data depicts one of two independent experiments. (FIG. 2G) Analysis of TIL of UA-fed mice, treated as depicted in FIG. 1J. Percentage of T_{SCM} within CD8+ (G; n=7), as well as TCF1 (H; n=7) PD1^{hi},Tim3^{hi} and CTLA4hi (I-K, all n=6) in the TIL CD8+ population are shown. Data are mean ± SD, statistical analysis was performed via two-sided t-test (*p < 0.05; **p < 0.01;, ***p <0.001.). Data depicts one of two independent experiments. (FIG. 2H) The same experiment as FIG. 2G with TCF1 (n=7) in the TIL CD8+ population. (FIG. 2I) The same experiment as FIG. 2G with PD1^{hi} (n=6) in the TIL CD8+ population. (FIG. 2J) The same experiment as FIG. 2G with Tim3^{hi} (n=6) in the TIL CD8+ population. (FIG. 2K) The same experiment as FIG. 2G with CTLA4hi (n=6) in the TIL CD8+ population. (FIG. 2L) Flow cytometry readout of ex vivo cytokine release of previously isolated TIL upon re-stimulation with PMA/ionomycin. Data are mean ± SD with n=7 per group. Statistical analysis was performed by two-sided t-test (**p < 0.01). One out of two independent experiments are shown. (FIG. 2M) Flow cytometry readout of ex vivo cytokine release of previously isolated TIL upon re-stimulation with PMA/ionomycin. Data are mean ± SD with n=7 per group. Statistical analysis was performed by two-sided t-test (**p < 0.01). One out of two independent experiments are shown.

UA treatment augments efficacy of adoptive cell transfer.

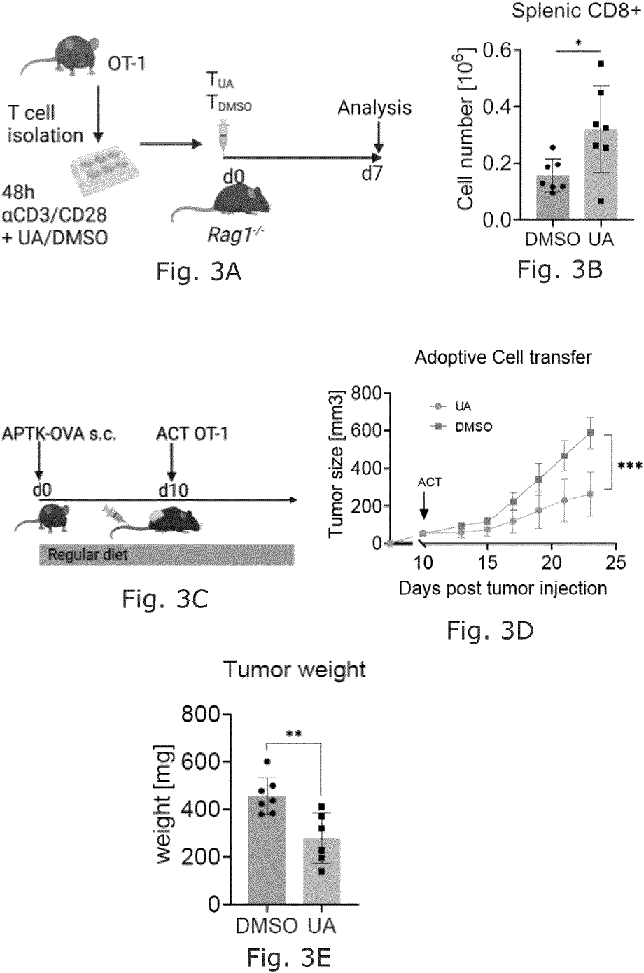

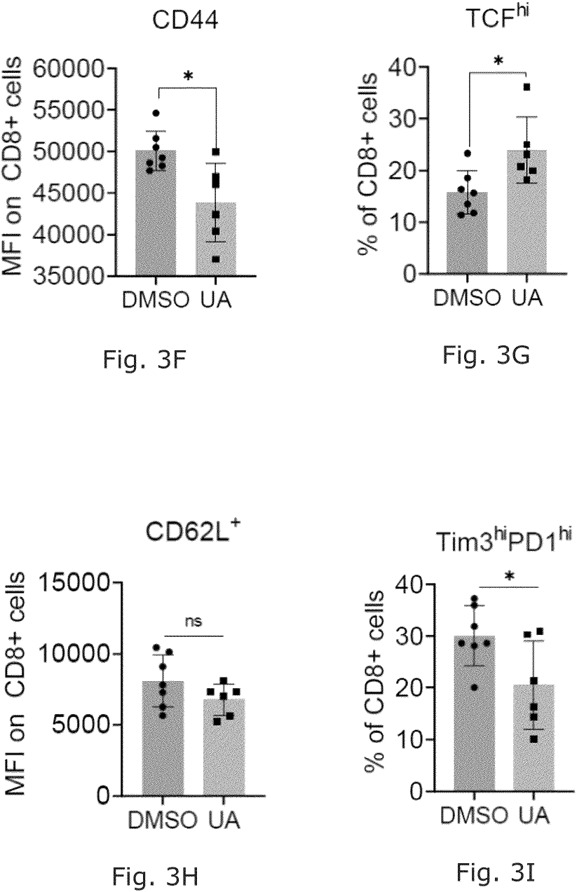

(FIG. 3A) Experimental setup of adoptive cell transfer in Rag1^{-/-} mice. CD3+ T-cells from OT1 donor mice were isolated and stimulated for 48 h in the presence of UA (resulting cells: T_{UA}) or DMSO (T_{DMSO}) control prior to transfer. (FIG. 3B) Flow cytometry analysis of CD8+ persistence in the spleens (n=7) of adoptively transferred Rag1^{-/-} mice. Data are mean ± SD with n=7 per group, statistical analysis was performed by two-sided t-test (**p < 0.01). (FIG. 3C) Experimental setup of adoptive cell transfer of OT-1 CD3+ cells in mice bearing APTK-OVA s.c. tumors (UA: n=6; DMSO: n=7). Ex vivo stimulation prior to transfer was performed as depicted in (A). Data are mean ± SD, ***p <0.001 by two-sided t-test. (FIG. 3D) Growth curve of s.c. APTK-OVA tumors treated as depicted in (C). Data are mean ± SD, ***p <0.001 by two-sided t-test. (FIG. 3E) Final tumor weight of s.c. transplanted APTK-OVA tumors treated by ACT of T_{UA} or T_{DMSO}. Data are mean ± SD, **p < 0.01 by two-sided t-test. (FIG. 3F) Analysis of TIL of ACT-treated Rag1^{-/-} mice carrying APTK-OVA tumors: Expression of CD44. All data depicts mean ± SD. Statistical analysis was performed by two-sided t-test (*p < 0.05; n.s. not significant). (FIG. 3G) Analysis of TIL of ACT-treated Rag1^{-/-} mice carrying APTK-OVA tumors: Expression of TCF1^{hi}. All data depicts mean ± SD. Statistical analysis was performed by two-sided t-test (*p < 0.05; n.s. not significant). (FIG. 3H) Analysis of TIL of ACT-treated Rag1^{-/-} mice carrying APTK-OVA tumors: Expression CD62L⁺. All data depicts mean ± SD. Statistical analysis was performed by two-sided t-test (*p < 0.05; n.s. not significant). (FIG. 3I) Analysis of TIL of ACT-treated Rag1^{-/-} mice carrying APTK-OVA tumors: percentage of exhausted Tim3^{hi}PD1^{hi} CD8+ cells. All data depicts mean ± SD. Statistical analysis was performed by two-sided t-test (*p < 0.05; n.s. not significant).

Urolithin A induces Pink1-dependent mitophagy in T cells.

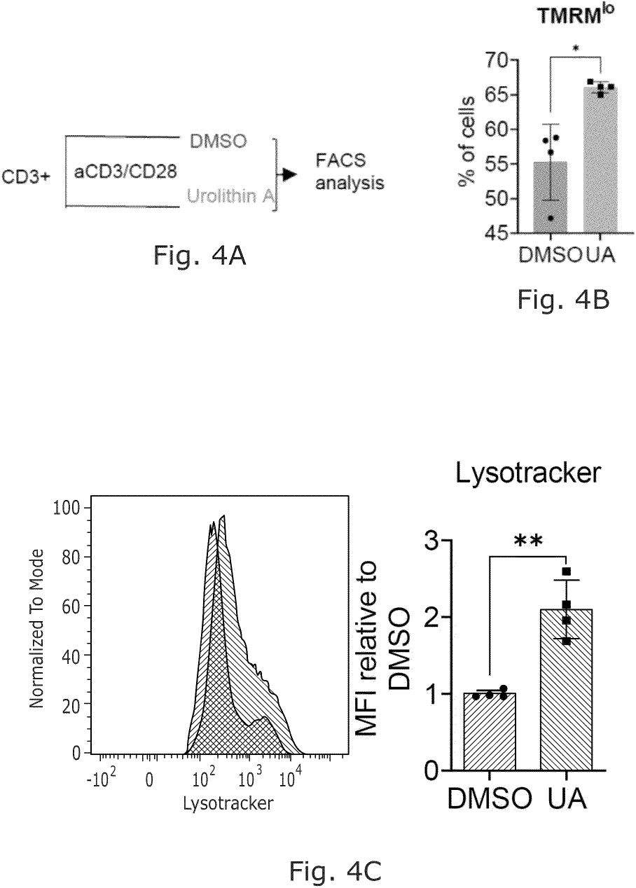

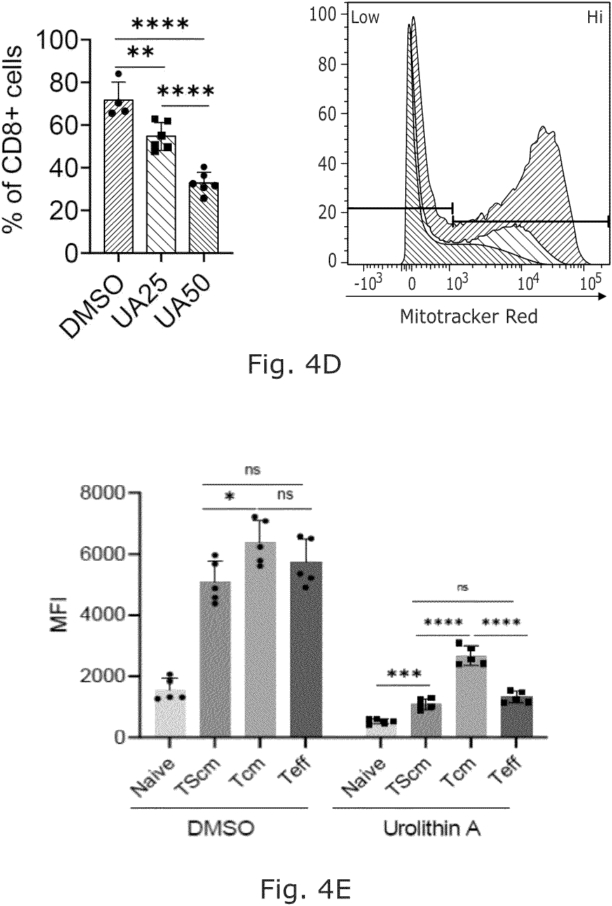

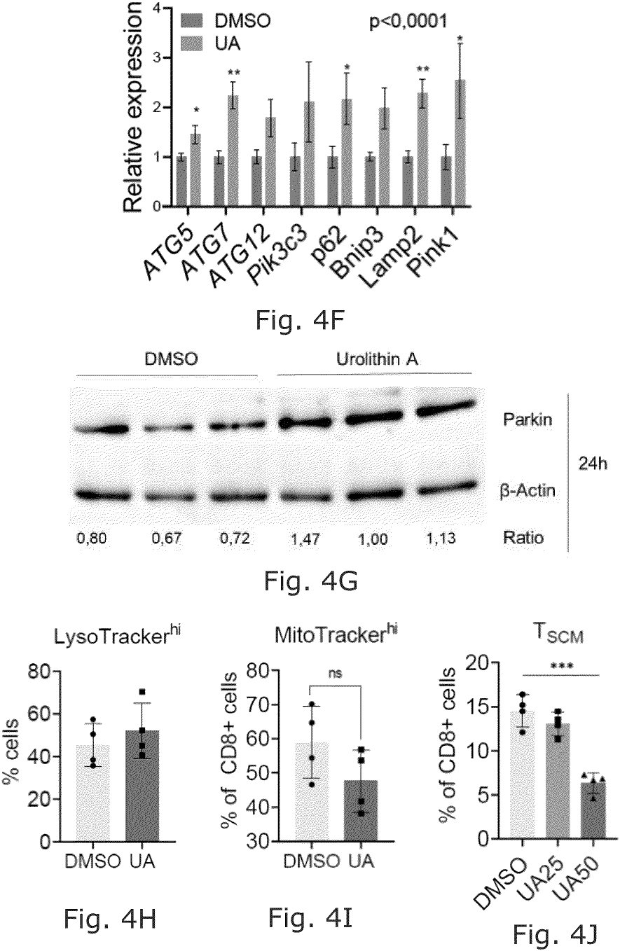

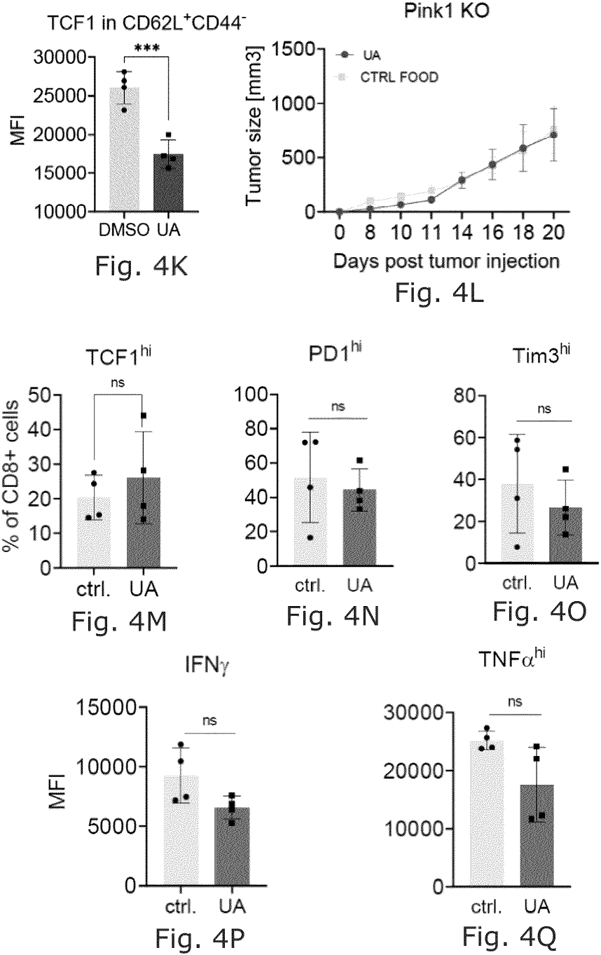

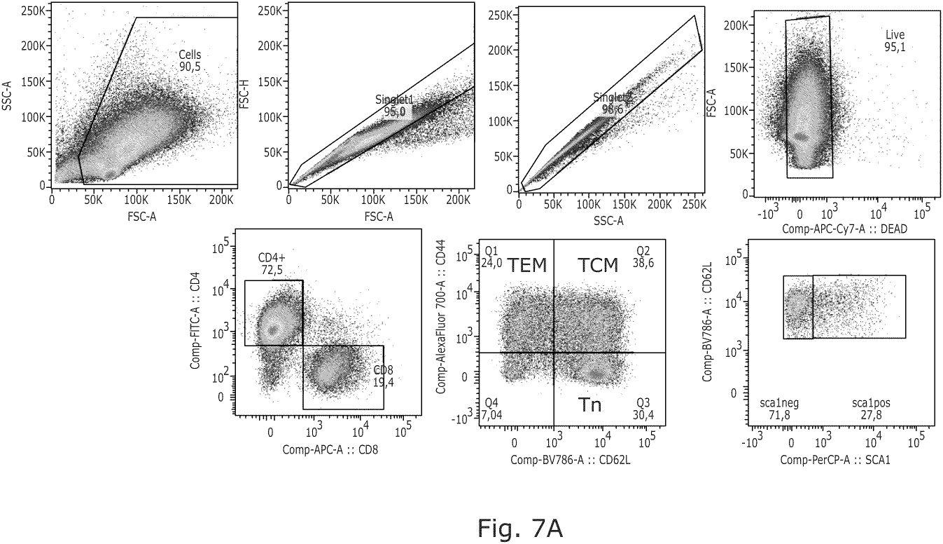

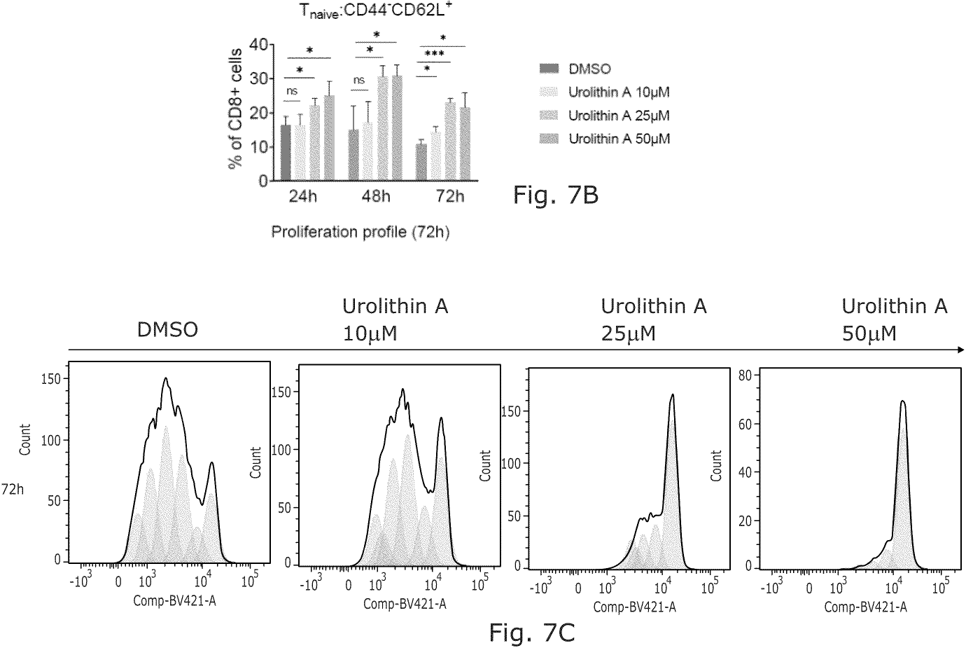

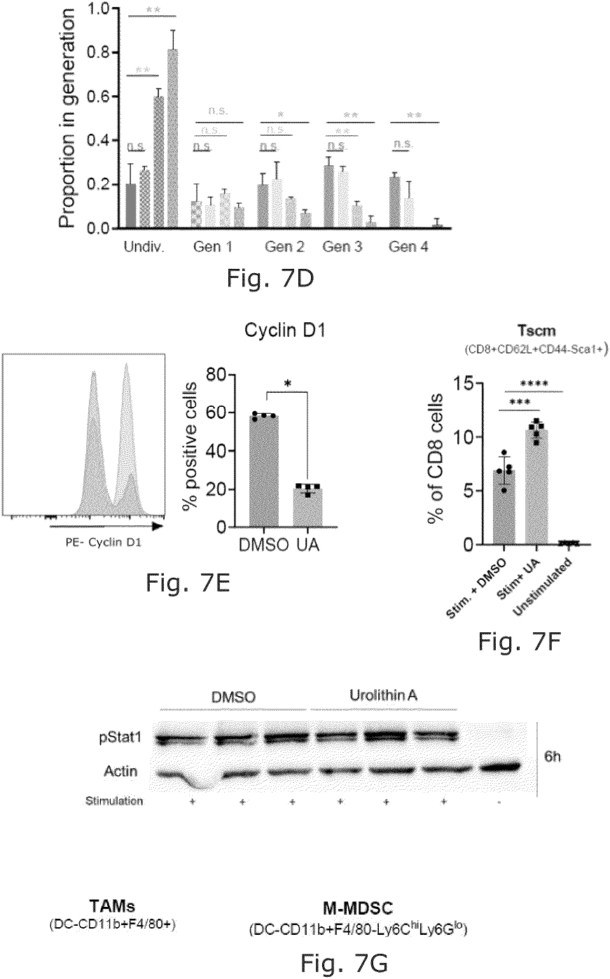

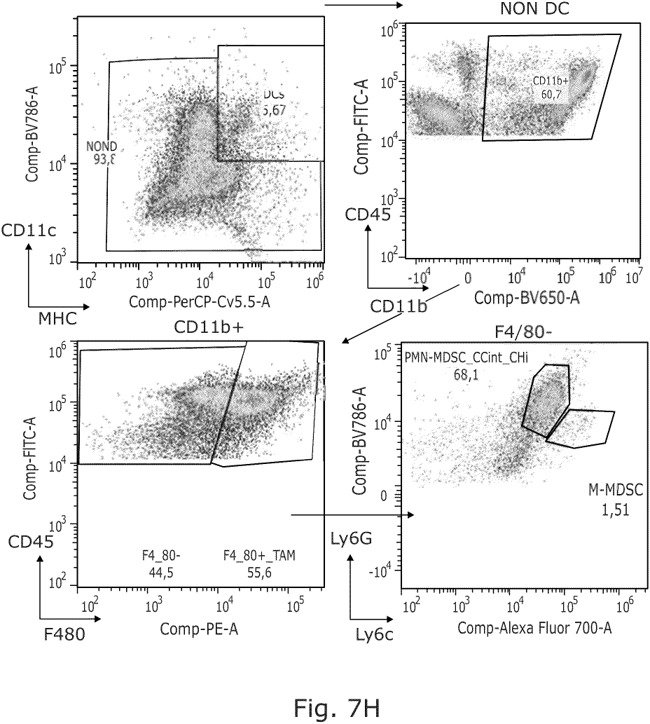

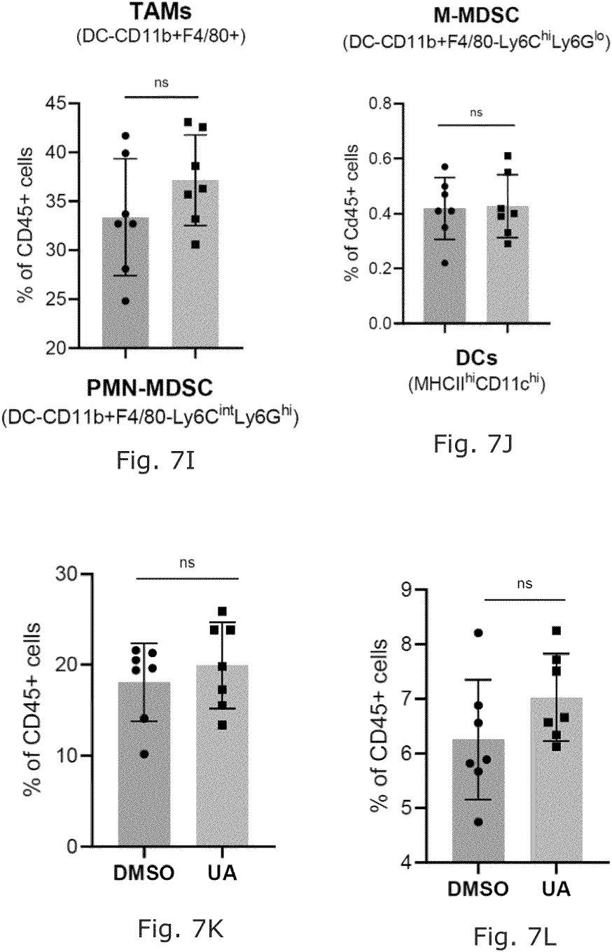

(FIG. 4A) Scheme of CD3+ T-cell activation and analysis for various timepoints in order to investigate mitophagy induction in vitro. (FIG. 4B) Analysis of mitochondrial membrane potential within CD8+ T cells, with TMRM^{lo} representing cells with low mitochondrial membrane potential. Analysis was performed six hours after stimulation with aCD/aCD28 stimulation beads at the presence of the indicated treatments. N=4 per group, *p < 0.05 by Mann Whitney U test. One of two independent experiments are shown. (FIG. 4C) Representative flow histogram and quantification of lysosome formation in CD8+ cells six hours after in vitro stimulation. MFI shown relative to DMSO controls, n=4. Data are normalised mean ± SD, statistical analysis by two-sided t-test. One out of two independent experiments are shown. (FIG. 4D) Mitotracker Red staining of in vitro stimulated CD8+ cells after 24 hours of Urolithin A (UA25 n=6; UA50 n=6) or vehicle control treatment (DMSO: n=4). A representative flow histogram depicting identification of Mitotracker^{hi} cells is shown. Data are mean ± SD, statistical analysis was performed by two-sided t-test (*p < 0.05; ***p <0.001; ****p < 0.0001). Experiment was repeated twice. (FIG. 4E) Subset-specific identification of Mitotracker red accumulation after 24 hours of in vitro stimulation either with vehicle control (DMSO) or 50 µM Urolithin A (n=5). Subsets were identified was depicted in the FIGS. 7-9. Data are mean ± SD, statistical analysis by two-sided t-test (* p<0.05, *** p<0.001 and **** p<0.0001). (FIG. 4F) qPCR analysis of various autophagy/mitophagy associated genes of in vitro activated CD3+ cells, 24 h after activation (n=3). Data are ± SEM, statistical analysis was performed by two-sided t-test (*p < 0.05,**p < 0.01). Overall p value is given for the comparison over the entire panel of genes after two-way ANOVA test. (FIG. 4G) Western blot of Parkin protein expression in UA or DMSO (n=3) treated cells after 24 h. Cells were stimulated at the presence of plate-bound aCD3/aCD28 antibodies. Ratio displays Parkin/GAPDH ratio. (FIG. 4H) CD8+ cells from Pink1^{-/-} mice display deficient mitophagy upon UA treatment in vitro: Lysosome formation in CD8+ of Pink1^{-/-} mice as assessed by Lysotracker staining 6h post stimulation at the presence of UA or DMSO (n=4). Data are mean ± SD, one out of two independent experiments are shown. (FIG. 4I) CD8+ cells from Pink1^{-/-} mice display deficient mitophagy upon UA treatment in vitro: Mitotracker red staining of Pink1^{-/-} CD8+ stimulated in vitro for 48 h, either in the presence of UA or DMSO (n=4). Percentage of Mitotracker^{hi} CD8+ cells are shown. Data are mean ± SD and acquired via flow cytometry. One out of two independent experiments are shown. Statistical analysis was performed by two-sided t-test (n.s., not significant). (FIG. 4J) CD8+ cells from Pink^{-/-} mice fail to reprogram to T_{SCM} after UA exposure in vitro: T_{SCM} formation in Pink1^{-/-} CD8+ cells 48 h after stimulation in the presence of UA (25 µM and 50 µM) and DMSO (n=4, respectively). Data are mean ± SD, statistical analysis was performed by two-sided t-test (***p <0.001). Data depicts results of three independent experiments. (FIG. 4K) Expression of TCF1 in naïve-like CD62L⁺CD44⁻CD8⁺ Pink1^{-/-} cells. Data are ± SD, depicted as MFI (***p <0.001 by two-sided t-test). (FIG. 4L) Growth curve of Pink1^{-/-} mice s.c. injected with APTK-organoids, fed UA-rich or control diet as depicted in FIG. 1J. n=4 per group. Data are mean ± SD. (FIG. 4M) Flow cytometry analysis of TIL in Pink ⁻^{/-} mice s.c. transplanted with APTK tumors, fed either UA or control diet. Quantification of TCF1^{hi} cells among CD8. (FIG. 4N) Quantification of PD1^{hi} cells among CD8. (FIG. 4O) Quantification of and Tim3^{hi} cells among CD8. (FIG. 4P) Ex vivo cytokine release assay. TIL of Pink1^{-/-} from FIG. 4L were stimulated ex vivo with PMA and lonomycin, in the presence of Brefeldin A for three hours. Data depicts percentage of TNFα^{hi} cells within the CD8+ compartment. All data are mean ± SD (n=4). Statistical analysis was performed by two-sided t-test (n.s. not significant). (FIG. 4Q) Data depicts percentage of IFN_{Y}^{hi} cells within the CD8+ compartment. All data are mean ± SD (n=4). Statistical analysis was performed by two-sided t-test (n.s. not significant).

UA induces T_{SCM} via cytosolic release of PGAM5 that drives Wnt signaling.

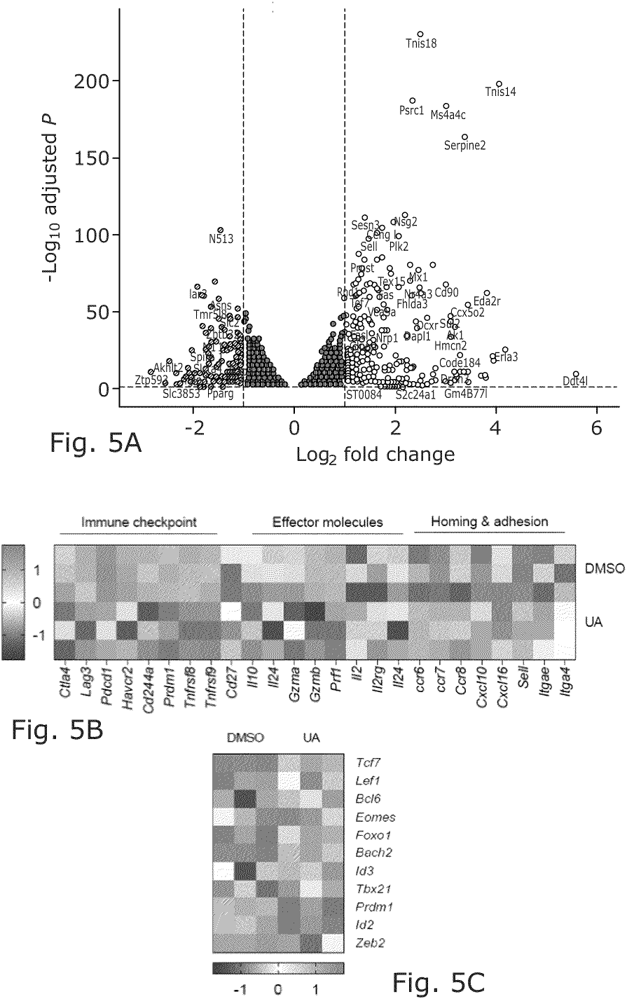

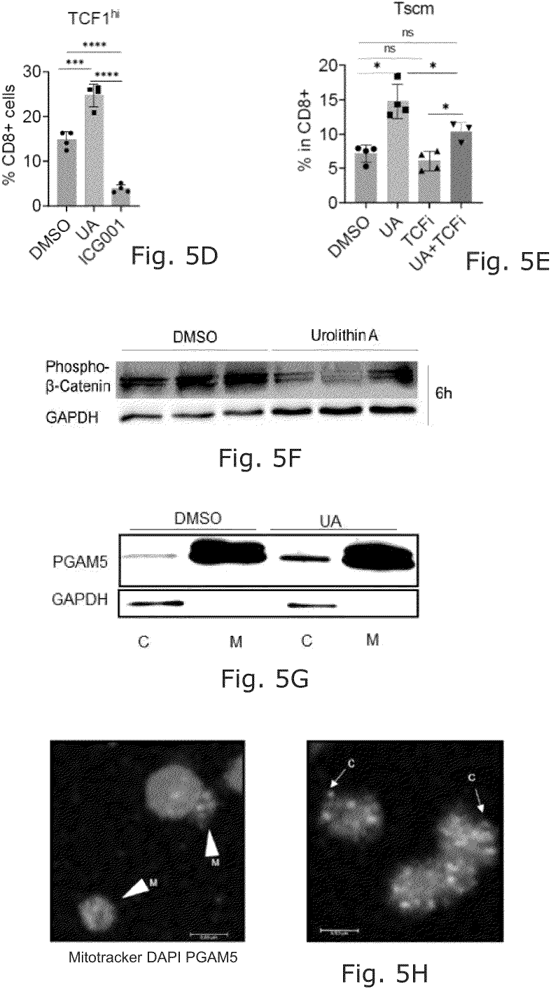

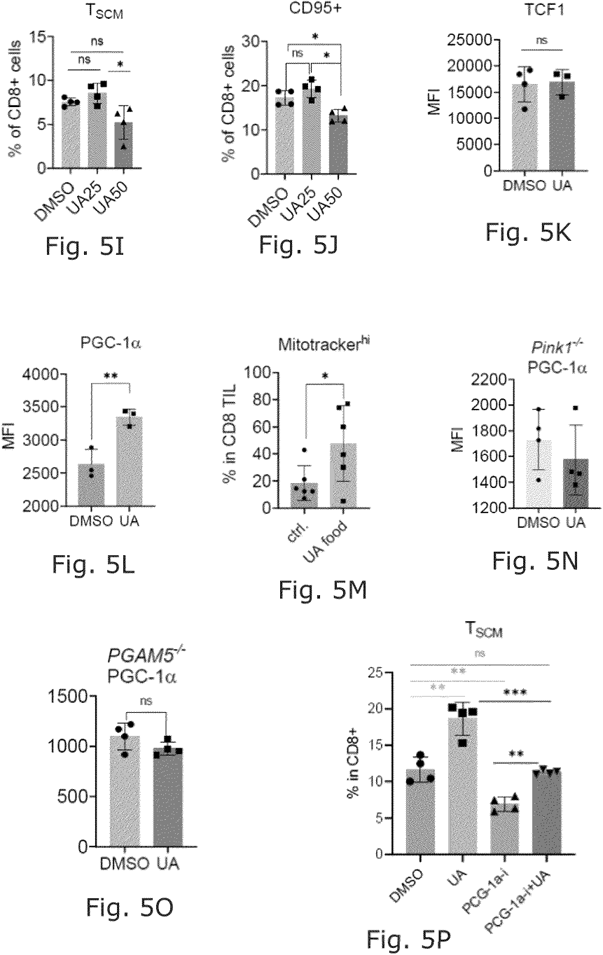

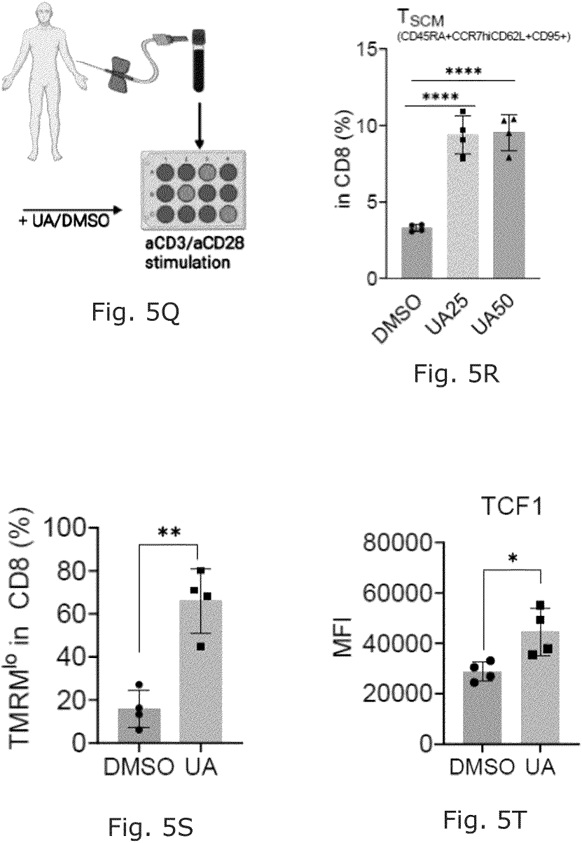

(FIG. 5A) Enhanced volcano plot of RNAseq of T cells stimulated for 48 h in vitro with aCD3/aCD28 at the presence of DMSO or Urolithin A. A log2 fold change of 1 and a p-value of p<0.05 were considered significant (red, significantly upregulated in UA treated cells; green, significantly upregulated in DMSO treated cells). (FIG. 5B) RNAseq expression heatmap of selected genes: Genes associated with immune checkpoints, effector molecules and genes encoding for leukocyte migration. (FIG. 5C) Expression of genes associated with T cell memory vs effector fate decisions. Data underwent z-score normalization for display (n=3 per group). (FIG. 5D) Uroltihin A induces high expression of TCF1, which abrogated by the TCF1 inhibitor ICG001. T cells were stimulated for 48 h in vitro at the presence of the indicated treatments. Data are mean ± SD (n=4/4/3), statistical analysis was performed by two-sided t-test (p***<0.01; p****0.0001). (FIG. 5E) Quantification of T_{SCM} formation within CD8+ cells after 48 h of in vitro stimulation under various treatment conditions, including TCF1 inhibition by ICG001 (DMSO, UA, TCF1i n=4; TCFi+UA n=3). Data are mean ± SD, statistical analysis was performed by two-sided t-test (*p < 0.05 ; n.s. not significant). One of two independent experiments are shown. (FIG. 5F) Western blot analysis of β-Catenin phosphorylation status in CD3+ cells stimulated via plate-bound aCD3/aCD28 antibodies for 6h. N=3 per group. (FIG. 5G) Cytosolic fractionation of CD3+ cells stimulated for six hours in the presence of Urolithin or DMSO control (c= cytosolic, m = mitochondrial fraction). Experiment was repeated twice.

(FIG. 5H) Immunofluorescence of PGAM5 (green) in CD3+ cells after in vitro stimulation with aCD3/aCD28 stimulation beads for six hours. Cells were stained with Mitotracker Red to display mitochondrial colocalisation in the DMSO control as opposed to cytosolic release in the UA sample (m, mitochondrial co-localization; c, cytoplasmic localization) Scale=5 um. (FIG. 5I) In vitro stimulation of PGAM5^{-/-} CD8+ cells: Percentage of T_{SCM} after 48 h of stimulation with aCD3/aCD28 stimulation beads in the presence of UA. All data depicts mean ± SD, n=4 per group. Statistics shown here depict unpaired, two-sided t-test and Mann-Whitney test (I; (DMSO vs UA25; DMSO vs UA50). *p < 0.05 ; n.s. not significant. (FIG. 5J) Percentage of CD95+ er 48 h of stimulation with aCD3/aCD28 stimulation beads in the presence of UA. (FIG. 5K) CD95+er 48 h of stimulation with aCD3/aCD28 stimulation beads in the presence of UA.

(FIG. 5L) UA induces PGC-1α expression in stimulated CD8+ cells. PGC-1α MFI at 48 h upon stimulation, as assessed by intracellular staining, is shown (n=3). One of two independent experiments are shown. Data are mean ± SD, statistical analysis was performed by two-sided t-test (p**<0.01). (FIG. 5M) Mitochondrial content of TIL CD8+ cells harvested from APTK-carrying mice, fed UA or control diet as depicted in FIG. 1J. Assessed by Mitotracker Red staining. Data are mean ± SD, *p < 0.05 by two-sided t-test. (FIG. 5N) Mitophagy and subsequent PGAM5 release drive PGC-1α upregulation. PGC-1α expression of CD8+ cells derived from Pink1^{-/-} KO mice upon 48 h stimulation in vitro in the presence of UA or DMSO control (n=4). Data are mean ± SD, statistical analysis was performed by two-sided t-test. Depicts one of two independent experiments. (FIG. 5O) Mitophagy and subsequent PGAM5 release drive PGC-1 α upregulation. PGC-1α expression of CD8+ cells derived from PGAM5⁻^{/-} mice upon 48 h stimulation in vitro in the presence of UA or DMSO control (n=4). Data are mean ± SD, statistical analysis was performed by two-sided t-test. FIG. 5P) PGC-1α inhibition reduces T_{SCM} formation in CD8+ cells upon in vitro stimulation. Cells were co-treated with DMSO, UA and an inhibitor of PGC-1α (PGC-1αi) as indicated (n=4). One of two independent experiments are shown. Data are mean ± SD, statistical analysis was performed by two-sided t-test (p**<0.01; ***p <0.001; n.s. not significant).(FIG. 5Q) Urolithin A induces T_{SCM} in human CD8+ cells: Schematic overview. PBMC were isolated from healthy donors, prior to T cell purification and in vitro stimulation via aCD3/aCD28 stimulation beads at the presence of UA or DMSO control. (FIG. 5R) Quantification of human T_{SCM} (n=4). Representative data of one out of five donors with the same result are shown. (FIG. 5S) Quantification of TMRM^{lo}CD8+ human cells 48 h after activation in the presence of UA or DMSO control (n=4). (FIG. 5T) TCF1 expression in human CD8 cells as assessed by flow cytometry (n=4). All data are mean ± SD; statistical analysis was performed by two-sided t-test (*p < 0.05; p**<0.01; p****0.0001).

Urolithin A promotes T_{SCM} facilitating generation of potent CAR-T_{SCM}.

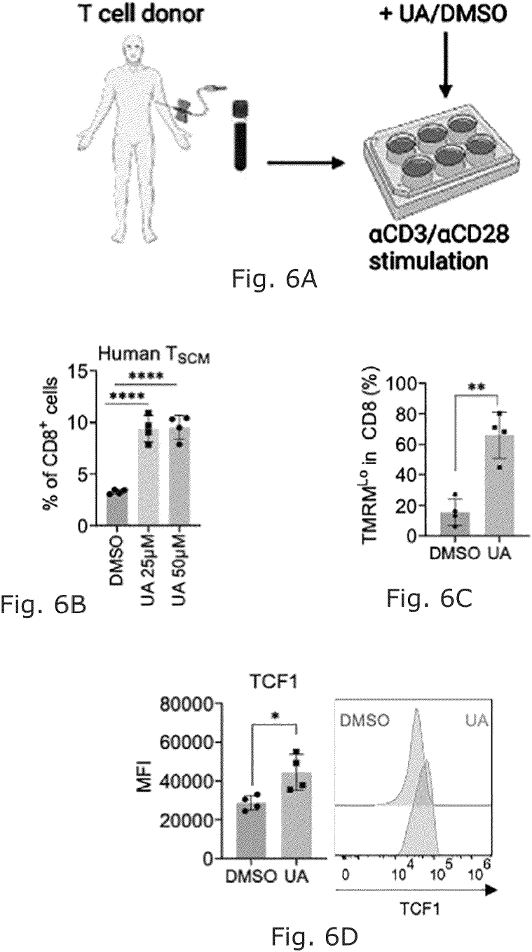

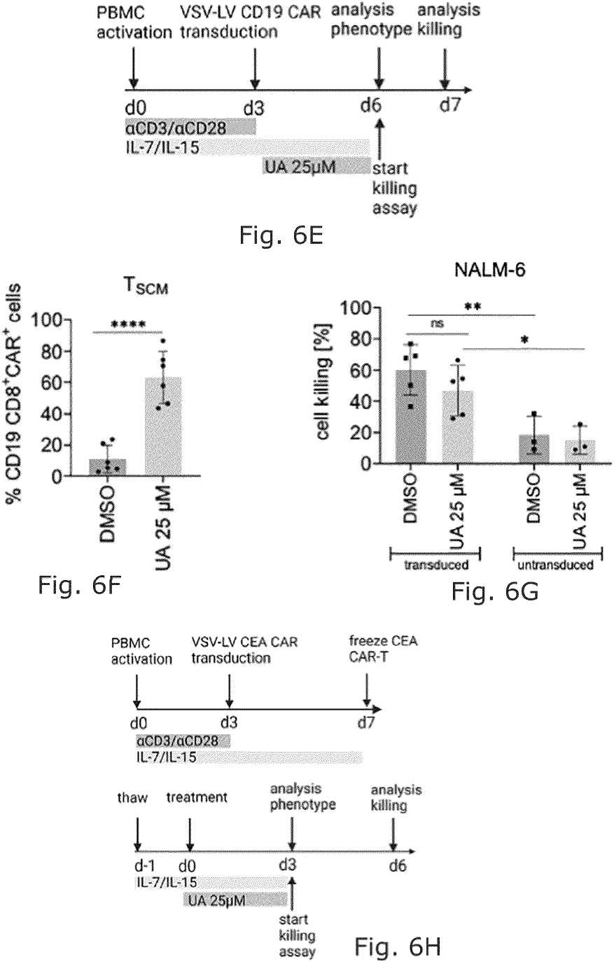

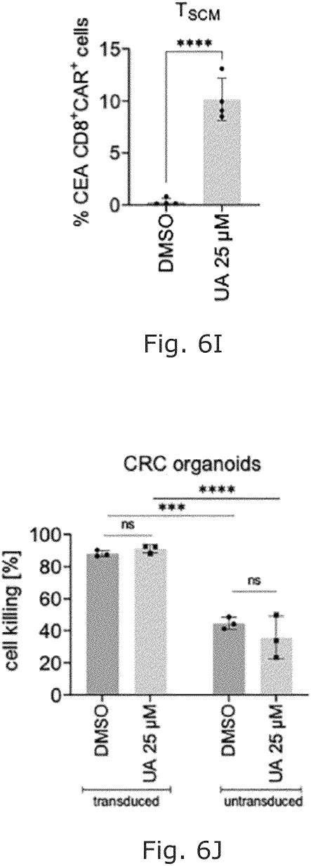

(FIG. 6A) Human PBMC were isolated from healthy donors, T cells were purified and stimulated ex-vivo with αCD3/αCD28 in the presence of UA (50 mM) or DMSO control.

(FIG. 6B) Frequency of human TSCM 48 h after stimulation as shown in (A). Data are mean ± SD, n=4, ****p<0.0001 by one-way ANOVA followed by Tukey’s multiple comparison test.

(FIG. 6C) Quantification of human TMRM^{lo}CD8⁺ T cells 48 h after stimulation in the presence of UA or DMSO control .Data are mean ± SD, n=4,**p<0.01 by two-sided t test.

(FIG. 6D) TCF1 expression in human CD8+ T cells 48 h after stimulation in the presence of UA or DMSO control. Data are mean ± SD, n=4,*p<0.05by two-sided t test. In (B), representative data of one out of five donors with comparable outcome are shown.

(FIG. 6E) Experimental layout of CD19-CART cell generation and expansion. PBMCS from healthy donors were expanded in the presence of IL-7/IL-15 for 3 days, followed by VSV-LV transduction. After 3 days of incubation, killing of Nalm-6 cells is assessed upon 24 h co-culture.

(FIG. 6F) Frequency of T_{SCM} within CD19⁻CAR⁺CD8⁺ cells after generation as depicted in (6E). Data are mean ± SD, n=6, ****p<0.0001 by two-sided t test. Data are pooled from three independent experiments.

(FIG. 6G) Killing potential of CD19-CARTcells. Percentage of dead NALM-6cells upon 24 h co-culture with untransduced or CAR-transduced T cells ± UA/DMSO is shown. Data are mean ±SD, n = 5/3 (transduced/untransduced); *p< 0.05, **p< 0.01, ****p< 0.0001 by two-way ANOVA followed Sidak’s multiple comparison test.

(FIG. 6H) Experimental layout of CEA-CART cell experiments. Following CAR gene transduction (upper panel), CART cells were frozen for subsequent experiments after thawing (lower panel).

(FIG. 6I) Frequency of T_{SCM} within CAR+CD8⁺cells specific for CEA Data are mean ±SD, n = 4, ****p < 0.0001 by two-sided t test. Data were pooled from two independent experiments.

(FIG. 6J) Killing potential of CEA-specific CART cells Percentage of dead CEA-expressing human CRC rganoids upon 72 h co-culture with untransducedor CAR-Transduced T cells ± UA/ DMSO is shown. Data are mea n± SD,n=3; ***p<0.001, ****p<0.0001,n.s.,not significant by two way ANOVA followed Sidak’ smultiple comparison test.

UA promotes naive-like T_{SCM}.

(FIG. 7A) Representative gating strategy for identifying naïve T cell (Tₙ; CD44-CD62L+), effector memory cell (T_{EM}; CD44⁻CD62L⁻), central memory cell (T_{CM;} CD44⁺CD62L⁺) and memory stem cell (T_{SCM}; CD44⁺CD62L⁺Sca1⁺) subsets within stimulated CD8+ cells. (FIG. 7B) Quantification of Tₙ within CD8+ stimulated in vitro by aCD3/CD28 beads for 48 h in the presence of different doses of UA (n=4). Data are mean ± SD, statistical analysis was performed by two-sided t-test (p*<0.05; ***p <0.001; n.s. not significant). (FIG. 7C) UA restricts T cell proliferation: Representative FACS micrograph of CTV staining after 72 h of in vitro stimulation. (FIG. 7D) UA restricts T cell proliferation: Statstical analysis of proliferation data obtained from (C) (n=3). Proportion in proliferative generation as assessed via FlowJo Software is shown (Undiv., undivided cells; Gen1, divided once). Data are mean ± SD, statistical analysis was performed by two-sided t-test (p*<0.05; ***p <0.001; n.s. not significant). One out of two independent experiments are shown. (FIG. 7E) UA restricts T cell proliferation: Representative flow micrograph and statistical analysis of Cyclin D1 expression at 48 h of stimulation +- Urolithin A (n=4). Representative of two independent experiments. Data are mean ± SD, statistical analysis was performed by Mann-Whitney-U test (p*<0.05).

(FIG. 7F) T_{SCM} arrise upon antigen encounter. Percentage of T_{SCM} present in CD8+ after 48 h stimulation via aCD3/CD28 beads +-UA or unstimulated CD8+ cells (n=5 per group). Data are mean ± SD, statistical analysis was performed by two-sided t-test (***p <0.001; ***p <0.0001).

(FIG. 7G) Western blot of T-Cell activation status as assessed by phosphorylation of STAT1 downstream of T cell activation at 6 h. Comparison of DMSO-treated, UA-treated and unstimulated cells is shown. (FIG. 7H) Oral UA treatment does not confer changes on non- T cell TIL in APTK tumors. Mice s.c transplanted with APTK tumors were treated as depicted in FIG. 1J. Representative gating strategy for identification of non- T cell TIL of interest. (FIG. 7I) Quantification of non-CD3+ TIL subsets as identified (n=7 per group): TAM. Data are mean ± SD, statistical analysis was performed by two-sided t-test (n.s., not significant). (FIG. 7J) Quantification of non-CD3+ TIL subsets as identified (n=7 per group): M-MDS. Data are mean ± SD, statistical analysis was performed by two-sided t-test (n.s., not significant). (FIG. 7K) Quantification of non-CD3+ TIL subsets as identified (n=7 per group): PMN-MDSC. Data are mean ± SD, statistical analysis was performed by two-sided t-test (n.s., not significant). (FIG. 7L) Quantification of non-CD3+ TIL subsets as identified (n=7 per group): DC. Data are mean ± SD, statistical analysis was performed by two-sided t-test (n.s., not significant).

UA-mediated mitophagy activates Wnt signaling.

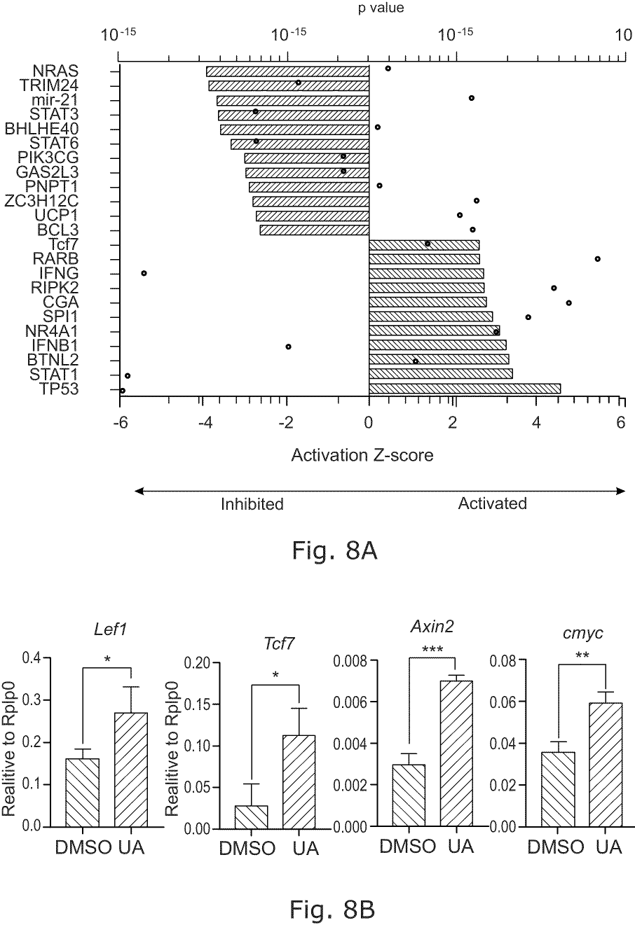

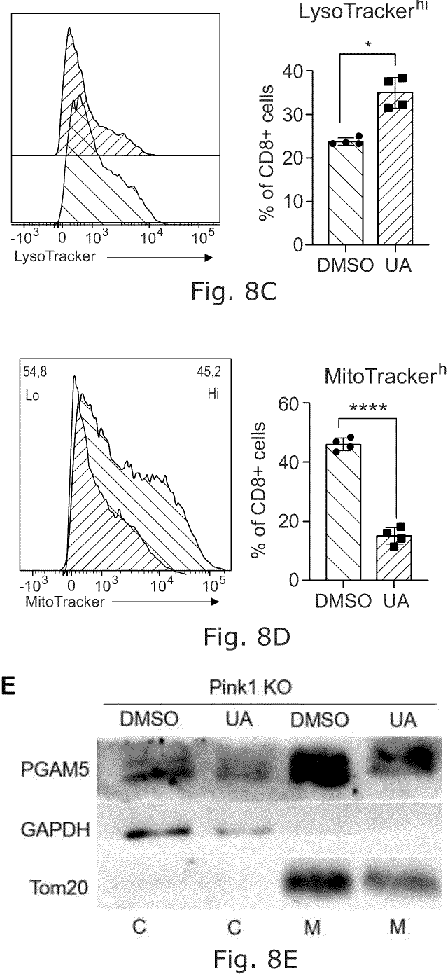

(FIG. 8A) Ingenuity pathway upstream regulator analysis of UA treated T cells. Assessed from RNAseq data. Plausible upregulation is depicted by z score, significance is shown on the overlying dot spot. Genes with a log2 fold change of 1 and p<0.05 were initially considered.

(FIG. 8B) qPCR analysis of selected Wnt target genes 24 h after stimulation (n=4). Data are mean ± SEM, statistical analysis was performed by two-sided t-test (* p<0.05, ** p<0.01, *** p<0.001). (FIG. 8C) CD8+ cells from PGAM5^{-/-} mice display an adequate mitophagy response upon UA treatment: Flow cytometry analysis of lysosome formation after six hours of in vitro stimulation via aCD3/aCD28 stimulation beads (n=4). (FIG. 8D) CD8+ cells from PGAM5^{-/-} mice display an adequate mitophagy response upon UA treatment: Mitotracker Red signal after 24 h in PGAM5^{-/-} CD8+ stimulated in vitro at the presence of UA or vehicle control. Congruent histograms are shown. Data are mean ± SD, statistical analysis was performed by two-sided t-test (* p<0.05, ** p<0.01, *** p<0.001). (FIG. 8E) Cytosolic fractionation Western blot of Pink1^{-/-} CD3+ cells 6 h after stimulation with plate-bound aCD3/aC28 antibodies in the presence of Urolithin A or DMSO control. One of two independent experiments is shown (c= cytosolic fraction, m=mitochondrial fraction).

UA induces T_{SCM} reprogramming in human CD8⁺ T cells.

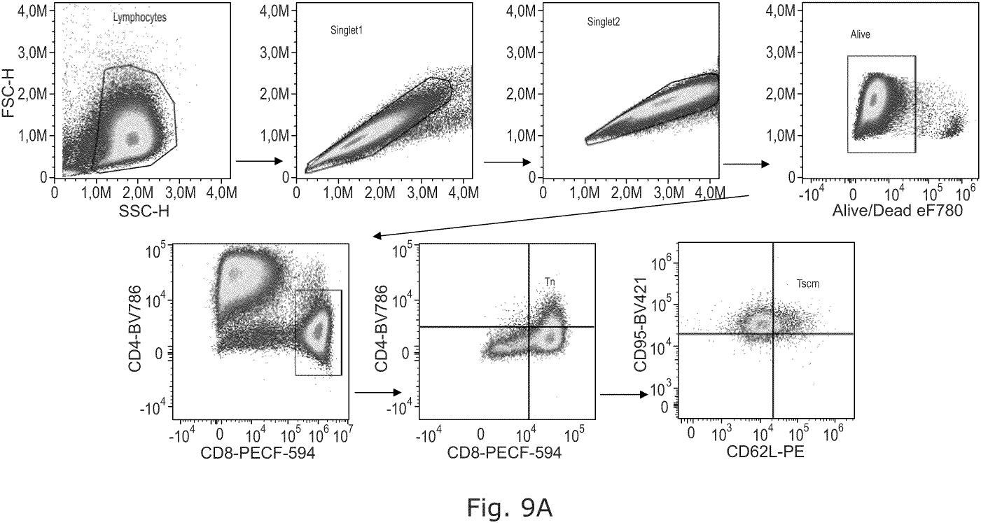

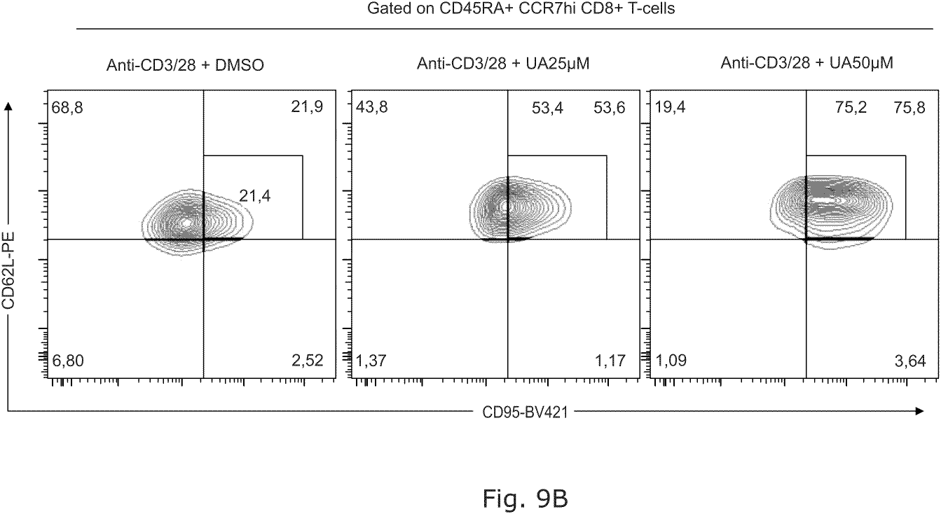

(FIG. 9A) Representative gating strategy for identification of human T_{SCM} (CD45RA⁺CCR7^{hi}CD62L⁺CD95⁺CD8⁺).

(FIG. 9B) Representative flow cytogram, displaying a dose-dependent increase of CD95^{hi}CD62L⁺ cells within the CD45RA⁺CCR7^{hi}CD8⁺ compartment.

### DETAILED DESCRIPTION

According to a first aspect of the invention, there is provided a urolithin for use in a method of overcoming or reversing of T-cell dysfunction.

According to a further aspect of the invention, there is provided a method of overcoming or reversing T-cell dysfunction using an effective amount of a urolithin.

According to a further aspect of the invention there is provided a urolithin for use in the manufacture of a medicament for use in a method of overcoming or reversing T-cell dysfunction.

In one aspect the T-cell dysfunction is T-cell exhaustion.

In a further aspect the T-cell dysfunction is T-cell exclusion.

In a further aspect the T-cell dysfunction is anergy.

In a further aspect the T-cell dysfunction is T-cell senescence.

According to a further aspect of the invention there is provided a urolithin for use in a method of enhancing T-cell fitness.

According to a further aspect of the invention, there is provided a method of enhancing T-cell fitness using an effective amount of a urolithin.

According to a further aspect of the invention there is provided a urolithin for use in the manufacture of a medicament for use in a method of enhancing T-cell fitness.

According to a further aspect of the invention, there is provided urolithin A for use in a method of enhancing T-cell fitness.

According to a further aspect of the invention, there is provided a method of enhancing T-cell fitness using an effective amount of urolithin A.

According to a further aspect of the invention there is provided urolithin A for use in the manufacture of a medicament for use in a method of enhancing T-cell fitness.

According to a further aspect of the invention there is provided a urolithin for use in a method of enhancing antigen presentation.

According to a further aspect of the invention, there is provided a method of enhancing antigen presentation using an effective amount of a urolithin.

According to a further aspect of the invention there is provided a urolithin for use in the manufacture of a medicament for use in a method of enhancing antigen presentation.

According to a further aspect of the invention, there is provided urolithin A for use in a method of enhancing antigen presentation.

According to a further aspect of the invention, there is provided a method of enhancing antigen presentation using an effective amount of urolithin A.

According to a further aspect of the invention there is provided urolithin A for use in the manufacture of a medicament for use in a method of enhancing antigen presentation.

In one embodiment, the enhancement of antigen presentation comprises upregulation of MHC molecules.

In another embodiment, the enhancement of antigen presentation comprises upregulation of MHC Class I molecules.

In a further embodiment, the enhancement of antigen presentation comprises upregulation of MHC Class II molecules.

In a further embodiment, the enhancement of antigen presentation comprises upregulation of MHC Class I and MHC Class II molecules.

In a further aspect the MHC molecules are upregulated on epithelia cells.

In a further aspect, the MHC molecules are upregulated on tumour epithelia cells.

In a further aspect the MHC Class I molecules are upregulated on epithelia cells.

In a further aspect, the MHC Class I molecules are upregulated on tumour epithelia cells.

In a further aspect the MHC Class II molecules are upregulated on epithelia cells.

In a further aspect, the MHC Class II molecules are upregulated on tumour epithelia cells.

In a further aspect the MHC Class I and Class II molecules are upregulated on epithelia cells.

In a further aspect, the MHC Class I and Class II molecules are upregulated on tumour epithelia cells.

According to a further aspect of the invention, there is provided a urolithin, for example urolithin A, for use in upregulating the level of MHC molecules.

According to a further aspect of the invention, there is provided a urolithin, for example, urolithin A, for use in the manufacture of a medicament for upregulating the level of MHC molecules.

According to a further aspect of the invention, there is provided a method for upregulating the level of MHC molecules using an effective amount of a urolithin, for example, urolithin A.

According to a further aspect of the invention, there is provided a urolithin, for example urolithin A, for use in upregulating the level of MHC molecules, wherein the MHC molecules comprise MHC Class I.

According to a further aspect of the invention, there is provided a urolithin, for example urolithin A, for use in the manufacture of a medicament for upregulating the level of MHC molecules, wherein the MHC molecules comprise MHC Class I.

According to a further aspect of the invention, there is provided a method for upregulating the level of MHC molecules, wherein the MHC molecules comprise MHC Class I, using an effective amount of a urolithin, for example urolithin A.

According to a further aspect of the invention, there is provided a urolithin, for example urolithin A, for use in upregulating the level of MHC molecules, wherein the MHC molecules comprise MHC Class II.

According to a further aspect of the invention, there is provided a urolithin, for example urolithin A, for use in the manufacture of a medicament for upregulating the level of MHC molecules, wherein the MHC molecules comprise MHC Class II.

According to a further aspect of the invention, there is provided a method for upregulating the level of MHC molecules, wherein the MHC molecules comprise MHC Class II, using an effective amount of a urolithin, for example urolithin A.

According to a further aspect of the invention, there is provided a urolithin, for example urolithin A, for use in upregulating the level of MHC molecules, wherein the MHC molecules comprise MHC Class I and Class II.

According to a further aspect of the invention, there is provided a urolithin, for example urolithin A, for use in the manufacture of a medicament for upregulating the level of MHC molecules, wherein the MHC molecules comprise MHC Class I and Class II.

According to a further aspect of the invention, there is provided a method for upregulating the level of MHC molecules, wherein the MHC molecules comprise MHC Class I and Class II, using an effective amount of a urolithin, for example urolithin A.

According to a further aspect of the invention there is provided a urolithin for use in a method for the expansion of T-cells.

According to a further aspect of the invention, there is provided a method for the expansion of T-cells using an effective amount of a urolithin.

According to a further aspect of the invention there is provided a urolithin for use in the manufacture of a medicament for use in a method for the expansion of T-cells.

According to a further aspect of the invention, there is provided urolithin A for use in a method of T-cell expansion.

According to a further aspect of the invention, there is provided a method of T-cell expansion using an effective amount of urolithin A.

According to a further aspect of the invention there is provided urolithin A for use in the manufacture of a medicament for use in a method of T-cell expansion.

In one embodiment, the T-cells are T stem cells.

In a further embodiment, the T-cells are T memory stem cells.

According to a further aspect, there is provided a T-cell population enriched in T-memory stem cells.

In one embodiment, the T-memory stem cell enriched population comprises CD8 positive cells.

In a further embodiment, the T-memory stem cells comprise between 10% to 80% of the CD8 positive cells, for example between 20% to 80% of the CD8 positive cells, for example between 30% to 80% of the CD8 positive cells, for example between 40% to 80% of the CD8 positive cells, for example between 40% to 70% of the CD8 positive cells, for example between 50% to 70% of the CD8 positive cells, for example between 55% to 65% of the CD8 positive cells, for example about 60% of the CD8 positive cells.

According to a further aspect of the invention, there is provided a urolithin for use in a method for the inhibition of T stem cell differentiation.

According to a further aspect of the invention, there is provided a urolithin for use in the manufacture of a medicament for use in a method for the inhibition of T stem cell differentiation.

According to a further aspect of the invention, there is provided a method for the inhibition of T stem cell differentiation using an effective amount of a urolithin.

In one embodiment, the inhibition is of T memory stem cells.

According to a further embodiment of the invention, there is provided a method of enhancing T memory stem cell numbers in a T-cell population comprising administering a urolithin to a population of T-cells, for example, a T-cell population isolated from spleen.

For the avoidance of doubt, a T-cell population may comprise cell types in addition to T-cells.

According to a further aspect of the invention there is provided a T-cell population comprising urolithin-treated T-cells.

According to a further aspect of the invention there is provided a urolithin-treated T-cell population as recited above, for use in therapy.

According to a further aspect of the invention there is provided a urolithin-treated T-cell population as recited above, for use in the treatment of a disease or conditions, for example, a disease or condition selected from cancer or an infectious disease or an autoimmune disease, for example, an infection disease selected from a bacterial infection, a viral infection or a parasitic infection.

According to a further aspect of the invention there is provided a urolithin for use in a method of T-cell expansion or inhibition of T stem cell differentiation, as set out above, for use in adoptive immunotherapy.

According to a further aspect of the invention there is provided a urolithin for use in a method of T-cell expansion or inhibition of T stem cell differentiation, as set out above, for use in adoptive T-cell transfer.

According to a further aspect of the invention, there is provided a T-cell population, of the invention, for use in a method of treating a disease, disorder or condition, for example, cancer, an autoimmune disease or an infectious disease.

According to a further aspect of the invention, there is provided a T-cell population, of the invention, for use in adoptive T-cell transfer for example, for the treatment of cancer, an autoimmune disease or an infectious disease.

According to a further aspect of the invention, there is provided a T-cell population, of the invention, for use in adoptive T-cell transfer for example, for the treatment of cancer, an autoimmune disease or an infectious disease.

According to a further aspect of the invention there is provided a urolithin for use in a method of treatment, wherein said method comprises the steps of:
(a) Isolating T cells from a patient, for example, a cancer patient;
(b) Culturing said T-cells ex-vivo; in the presence of a urolithin; and
(c) Administration of the urolithin-treated cells to the patient.

According to a further aspect of the invention there is provided a urolithin for use in a method of treating a disease, disorder or condition, for example, cancer, an autoimmune disease or an infectious disease, wherein said method comprises the steps of:
(d) Isolating T cells from a patient, for example, a patient suffering from cancer;
(e) Culturing said T-cells ex-vivo; in the presence of a urolithin; and
(f) Administration of the urolithin-treated cells to the patient.

According to a further aspect of the invention there is provided a composition comprising T-cells produced by a method as described above.

According to a further aspect of the invention there is provided a composition comprising T-cells obtained by a method as described above.

According to a further aspect of the invention there is provided a composition comprising T-cells obtainable by a method as described above.

According to a further aspect of the invention there is provided a composition comprising T-cells produced by a method as described above for use in the treatment of a disease or condition, for example, where the disease or condition is cancer or an infectious disease or an autoimmune disease.

According to a further aspect of the invention, there is provided a urolithin for use in a method of augmentation of immune-mediated anti-tumour memory.

According to a further aspect of the invention, there is provided a urolithin for use in a method of augmentation of immune-mediated anti-tumour memory after T cell expansion for use in adoptive immunotherapy.

According to a further aspect of the invention, there is provided a urolithin for use in a method of enhancing CD8+ve T-cell dependent anti-tumour immunity.

According to a further aspect of the invention, there is provided a urolithin for use in a method of enhancing CD8+ve T-cell dependent anti-tumour immunity, wherein the method comprises:
(a) Isolating a T-cell population from a patient, for example, a population comprising CD3-positive T-cells;
(b) Administering a urolithin to the T cell population, ex vivo; and
(c) Administering the treated T-cell population to the patient.

Adoptive stem cell transfer benefits especially from minimally differentiated cells due to their improved survival and long term potential to generate unexhausted effector cells (Luca Mo et al(2011) Nature 597, 544-548).

According to a further aspect of the invention, there is provided a urolithin for use in a method of preparing a T-cell population which is enriched in T memory stem cells, comprising administering a urolithin, for example, urolithin A, to a sample of T-cells obtained from a subject.

According to a further aspect of the invention, there is provided a method of preparing a T-cell population which is enriched in T memory stem cells, comprising administering a urolithin, for example, urolithin A, to a sample of T-cells obtained from a subject.

According to a further aspect of the invention, there is provided a urolithin, for example, urolithin A, for use in the manufacture of a medicament for use in a method of preparing a T-cell population which is enriched in T memory stem cells, comprising administering a urolithin to a sample of T-cells obtained from a subject.

According to a further aspect of the invention, there is provided a urolithin for use in a method of preparing a T-cell population which is enriched in T memory stem cells, comprising administering a urolithin, for example, urolithin A, to a sample of T-cells.

According to a further aspect of the invention, there is provided a method of preparing a T-cell population which is enriched in T memory stem cells, comprising administering a urolithin, for example, urolithin A, to a sample of T-cells.

According to a further aspect of the invention, there is provided a urolithin, for example, urolithin A, for use in the manufacture of a medicament for use in a method of preparing a T-cell population which is enriched in T memory stem cells, comprising administering a urolithin to a sample of T-cells.

Methods of the invention can be used in methods of CAR-T cell generation (T cells carrying an engineered chimeric antigen receptor (CAR)), for use in adoptive cell therapy.

Therefore, according to a further aspect of the invention, there is provided a method of preparing a T memory stem cell enriched CAR-T cell population comprising administering a urolithin, for example, urolithin A, to a population of CAR gene transfected T cells, for example, for use in adoptive cell therapy.

According to a further aspect of the invention, there is provided a urolithin, for example, urolithin A, for use in a method of preparing a T memory stem cell enriched CAR-T cell population comprising administering a urolithin to a population of CAR gene transfected T cells, for example, for use in adoptive cell therapy.

According to a further aspect of the invention, there is provided a urolithin, for example, urolithin A, for use in the manufacture of a T memory stem cell enriched CAR-T cell population comprising administering a urolithin to a population of CAR gene transfected T cells, for example, for use in adoptive cell therapy.

According to a further aspect of the invention, there is provide a method of producing a population of urolithin-treated CAR-T cells, wherein said method comprises the steps of:
(a) Obtaining T cells from a subject;
(b) Transfecting the T cells with a CAR (chimeric antigen receptor) gene, to prepare CAT-T cells;
(c) Administering a urolithin, for example, urolithin A to the CAR-T cells, to produce a population of urolithin-treated CAR-T cells;

According to a further aspect of the invention, there is provided a method of adoptive cell therapy, wherein said method comprises the steps of:
(d) Obtaining T cells from a subject;
(e) Transfecting the T cells with a CAR (chimeric antigen receptor) gene, to prepare CAT-T cells;
(f) Administering a urolithin to the CAR-T cells, to produce a population of urolithin-treated CAR-T cells;
(g) Administering the urolithin-treated CAR-T cells to the subject or a different subject.

A method for producing T cells expressing a chimeric antigen receptor (CAR- T cells), the method comprising: (i) culturing a first population of CAR- transfected T-cells in a medium comprising a urolithin, for example, urolithin A, to produce a second population of CAR-transfected T cells.

According to a further aspect of the invention, there is provided a population of urolithin-treated, for example, urolithin A-treated, T cells.

According to a further aspect of the invention, there is provided a population of urolithin-treated, for example, urolithin A-treated, CAR-T cells.

According to a further aspect of the invention, there is provided a T memory stem cell enriched CAR-T cell population, obtainable by treatment of a CAR-T cell population by the administration of a urolithin, for example, urolithin A.

According to a further aspect of the invention, there is provided a chimeric antigen receptor T (CAR- T) cell population for use in adoptive cell therapy, comprising T cells engineered to express a chimeric antigen receptor (CAR) polypeptide treated with a urolithin, for example, urolithin A.

A method of treating a disease, for example, cancer in a subject, the method comprising administering urolithin-treated CAR T-cells, for example, urolithin A-treated, CAR-T cells.

A urolithin-treated, for example, urolithin A-treated, CAR-T cell population for use in the treatment of a disease, for example, cancer.

A urolithin-treated, for example, urolithin A-treated, CAR-T cells population for use in the manufacture of a medicament for use in the treatment of cancer.

An adoptive cell therapy method for treating cancer, comprising the step of administering CAR-T cells to a subject suffering from cancer, wherein the CAR-T cells have been pre-treated with a urolithin, for example, urolithin A.

CAR-T cells for use in an adoptive cell therapy method for treating cancer, comprising the step of administering CAR-T cells to a subject suffering from cancer, wherein the CAR-T cells have been pre-treated with a urolithin, for example, urolithin A.

CAR-T cells for use in the manufacture of a medicament for use in an adoptive cell therapy method for treating cancer, comprising the step of administering CAR-T cells to a subject suffering from cancer, wherein the CAR-T cells have been pre-treated with a urolithin, for example, urolithin A.

According to a further aspect of the invention, there is provided a urolithin for a use or in a method of the invention with the proviso that use or method is substantially in the absence of nicotinamide riboside.

According to a further aspect of the invention, there is provided a urolithin for a use or in a method of the invention with the proviso that use or method is in the absence of nicotinamide riboside.

According to a further aspect of the invention, there is provided a urolithin for a use or in a method of the invention with the proviso that use or method is substantially in the absence of vitamin B12.

According to a further aspect of the invention, there is provided a urolithin for a use or in a method of the invention with the proviso that use or method is in the absence of vitamin B12.

According to a further aspect of the invention, there is provided a urolithin for a use or in a method of the invention with the proviso that use or method is substantially in the absence of manganese salts, for example manganese chloride.

According to a further aspect of the invention, there is provided a urolithin for a use or in a method of the invention with the proviso that use or method is in the absence of manganese salts, for example, manganese chloride.

According to a further aspect of the invention, there is provided a urolithin for a use or in a method of the invention with the proviso that use or method is substantially in the absence of nicotinamide riboside and manganese salts, for example manganese chloride.

According to a further aspect of the invention, there is provided a urolithin for a use or in a method of the invention with the proviso that use or method is in the absence of nicotinamide riboside and manganese salts, for example manganese chloride.

According to a further aspect of the invention, there is provided a urolithin for a use or in a method of the invention with the proviso that use or method is substantially in the absence of manganese salts, for example manganese chloride, and vitamin B12.

According to a further aspect of the invention, there is provided a urolithin for a use or in a method of the invention with the proviso that use or method is in the absence of manganese salts, for example manganese chloride and vitamin B12.

According to a further aspect of the invention, there is provided a urolithin for a use or in a method of the invention with the proviso that use or method is substantially in the absence of nicotinamide riboside, manganese salts, for example manganese chloride, and vitamin B12.

According to a further aspect of the invention, there is provided a urolithin for a use or in a method of the invention with the proviso that use or method is in the absence of nicotinamide riboside, manganese salts, for example manganese chloride and vitamin B12.

In uses and methods of the invention, a urolithin may be combined with an immunotherapy treatment, such as an immune checkpoint blockage therapy.

In uses and methods of the invention, urolithin or a T-cell population treated with a urolithin may be combined with an immunotherapy treatment, such as an immune checkpoint blockage therapy.

In uses and methods of the invention, urolithin may be combined with an agent which modulates immune-inhibitory proteins, such as PD-1 or PD-L1

In uses and methods of the invention, urolithin or a T-cell population treated with a urolithin may be combined with an agent which modulates immune-inhibitory proteins, such as PD-1 or PD-L1

In uses and methods of the invention comprising combinations with urolithin, the components of the combination may be administered by separate, sequential or simultaneous administration.

In uses and methods of the invention comprising combinations with urolithin, or a T-cell population treated with a urolithin, the components of the combination may be administered by separate, sequential or simultaneous administration.

In uses and methods of the invention, combinations may be provided as a composition, for example a pharmaceutical composition.

According a further aspect of the invention there is provided a composition, for example, a pharmaceutical composition, comprising a combination of a urolithin and an immunotherapy treatment for use in a method or use of the invention.

According a further aspect of the invention there is provided a composition, for example, a pharmaceutical composition, comprising a combination of a urolithin or a T-cell population treated with a urolithin, and an immunotherapy treatment for use in a method or use of the invention.

According a further aspect of the invention there is provided a composition, for example, a pharmaceutical composition, comprising a combination of a urolithin and an immune checkpoint blockage therapy for use in a method or use of the invention.

According a further aspect of the invention there is provided a composition, for example, a pharmaceutical composition, comprising a combination of a urolithin, or a T-cell population treated with a urolithin, and an immune checkpoint blockage therapy for use in a method or use of the invention.

According a further aspect of the invention there is provided a composition, for example, a pharmaceutical composition, comprising a combination of a urolithin and an agent which modulates immune-inhibitory proteins for use in a method or use of the invention.

According a further aspect of the invention there is provided a composition, for example, a pharmaceutical composition, comprising a combination of a urolithin, or a T-cell population treated with a urolithin, and an agent which modulates immune-inhibitory proteins for use in a method or use of the invention.

In one embodiment, the immune checkpoint blockage therapy is selected from: a PD-1 antagonist, an anti-CTLA4 therapy, a CD28 antagonist, a B7 ligand antagonist (for example, an antagonist of B7-1 (CD80) or B7-2 (CD86)), a CD27 antagonist, a CD40 antagonist, a CD40 Ligand, an OX40 antagonist, a GITR antagonist, a CD137 antagonist and/or a 41-BB-1 antagonist.

In one embodiment, the immune checkpoint blockage therapy is selected from: a PD-1 antagonist, an anti-CTLA4 therapy, and a CD28 antagonist, and/or a B7 ligand antagonist (for example, an antagonist of B7-1 (CD80), B7-2 (CD86), a CD27 antagonist, a CD40 antagonist, a CD40 Ligand, an OX40 antagonist, a GITR antagonist, a CD137 antagonist and/or a 41-BB- I antagonist.

In a further embodiment, the PD-1 antagonist is an anti-PD-1 antibody or functional part thereof. Examples of anti-PD-1 antibodies include pembrolizumab, nivolumab (BMS-936558), cemiplimab and pidilizumab.

In a further embodiment, the PD-1 antagonist is an anti-PD-L1 antibody or functional part thereof. Examples of anti-PD-L1 antibodies include avelumab, atezolizumab (MPDL3280A) and durvalumab.

In another embodiment, the PD-1 antagonist is a fusion protein such as AMP-224 (a recombinant B7-DC Fc-fusion protein composed of the extracellular domain of the PD-1 ligand programmed cell death ligand 2 (PD-L2, B7-DC) and the Fc region of human immunoglobulin (Ig) G₁).

In a further embodiment, the immune checkpoint blockage therapy is an anti-CTLA4 therapy. Examples of anti-CTLA4 therapy include: ipilimumab and tremelimumab

The combination of the invention is useful for the treatment of diseases wherein there is T-cell dysfunction. Examples of such diseases include cancer and infectious diseases and autoimmune diseases.

Examples of suitable cancers include: solid tumours, including HIV-associated metastatic solid tumours.

Examples of suitable cancers include: bladder cancer, B-cell lymphoma such as Hodgkin’s lymphoma, T-cell lymphoma, T-cell acute lymphoblastic leukemia, acute lymphoblastic leukemia, chrome lymphoblastic leukemia, acute myelogenous leukemia, chronic myelogenous leukemia, erythroleukemia, triple negative breast cancer, breast cancer, ovarian cancer, melanoma including paediatric melanoma, lung cancer such as squamous cell lung carcinoma and non small-cell lung cancer, pancreatic cancer, glioblastoma, colorectal cancer, head and neck cancer such a head and neck squamous cell carcinoma, cervical cancer, prostate cancer, liver cancer, oral squamous cell carcinoma, skin cancer, medulloblastoma, hepatocellular carcinoma, intrahepatic and extrahepatic cholangiocarcinoma, desmoid tumours, soft tissue sarcoma, adenoid cystic carcinoma, urothelial cancer, renal cancer, hepatocellular cancer, skin cancer, such as Merkel cell carcinoma, gastric cancer and gastroesophageal cancer.

In one embodiment, the cancer is colorectal cancer.

In one embodiment, a suitable cancer is a microsatellite instability high (MSI-H) or mismatch repair deficient (dMMR) solid tumour.

Examples of infectious diseases include: viral, bacterial, fungal and parasitic infection.

Examples of viral infections include: lymphocytic choriomeningitis virus (LCMV), HIV, hepatitis B virus (HBV), or hepatitis C virus (HCV).

Examples of bacterial infections include: Helicobacter pylori, Mycobacterium tuberculosis (MTB), sepsis (gram positive bacteria such as staphylococcus) and nosocomial (hospital acquired infections, such as C. difficile).

Examples of parasitic infections include: helminth parasites, including Schistosoma mansoni, Schistosoma japonicum, Schistosoma haematobium, Fasciola hepatica, and Heligmosomoides polygyrus, Leishmania such as Leishmania donovani, Leishmania chagasi and Leishmania Mexicana, Plasmodium, such as Plasmodium berghei and Plasmodium falciparum, Toxoplasma, such as Toxoplasma gondii.

Examples of fungal infections include: Candidiasis, Aspergillosis and Cryptococci.

Examples of infectious diseases include, but are not limited to HIV, Hepatitis (A, B, & C), Influenza, Herpes, Giardia, Malaria, Leishmania, Staphylococcus aureus and Pseudomonas Aeruginosa.

### Additional/Combination Therapy

The embodiments of the invention described herein may be combined with further therapies, such as radiation therapy and/or one or more additional therapeutic agents.

In some embodiments, the composition further comprises one or more therapeutic agents comprising, anti-cancer agents, anti-viral agents, anti-inflammatory agents, and or adjuvants.

In one embodiment the further therapy is radiation therapy.

In one mode, the radiation therapy is fractionated radiation therapy. In one embodiment, the fractionated radiation therapy comprises from 2 to 7 fractions. In another embodiment, the fractionated radiation therapy comprises from 3 to 6 fractions. In another embodiment, the fractionated radiation therapy comprises from 4 to 5 fractions. In one mode, the fractionated radiation therapy comprises 2, 3, 4, 5, 6, or 7 fractions. In one embodiment, the fractionated radiation therapy comprises 5 fractions.

In one mode, the radiation therapy fractions are administered in sequential days. In one mode, radiation therapy may include more than one dose on a day and/or doses on sequential days. In one mode, the radiation therapy fractions are administered on day 1, day 2, day 3, day 4, and day 5. In another mode, the radiation therapy comprises about 10 Gy in 5 fractions (i.e., 2 Gy on each of 5 days).

Other fractionation schedules may be employed including accelerated fractionation (treatment given in larger daily or weekly doses to reduce the number of weeks of treatment), hyperfractionation (smaller doses of radiation given more than once a day), or hypofractionation (larger doses given once a day or less often to reduce the number of treatments).

The radiation therapy may be x-rays, gamma rays, or charged particles. The radiation therapy may be external-beam radiation therapy or internal radiation therapy (also called brachytherapy). Systemic radiation therapy, using radioactive substances, such as radioactive iodine, may also be employed.

External-beam radiation therapy includes 3D conformational radiation therapy, intensity-modulated radiation therapy, image-guided radiation therapy, tomotherapy, stereotactic radiosurgery, proton therapy, or other charged particle beams.

In another embodiment, the one or more additional therapeutic agents, includes, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

Specific examples of such additional therapeutic agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti- inflammatory drugs (e.g., aspirin, ibuprofen, diclofenac, and COX-2 inhibitors), pain relievers, leukotriene antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, anti-histamines, anti-malarial agents (e.g., hydroxychloroquine), anti-viral agents (e.g., nucleoside analogs (e.g., remdesivir, zidovudine, acyclovir, ganciclovir, vidarabine, idoxuridine, trifluridine, and ribavirin), foscamet, amantadine, rimantadine, saquinavir, indinavir, ritonavir, and AZT) and antibiotics (e.g., dactinomycin (formerly actinomycin), bleomycin, erythromycin, penicillin, mithramycin, and anthramycin (AMC)).

Any therapy which is known to be useful, or which has been used or is currently being used for the treatment of a disease state associated with inhibition of T-cell activation can be used in combination with the invention. See, e.g., Gilman et al, Goodman and Gilman’s: The Pharmacological Basis of Therapeutics, 13th ed., McGraw-Hill, New York, 2017; The Merck Manual of Diagnosis and Therapy, Robert S. Porter, M.D. et al. (eds.), 20th Ed., Merck Sharp & Dohme Research Laboratories, Rahway, NJ, 2018; Cecil Textbook of Medicine, 25th Ed., Goldman and Schafer (eds.), Elsevier, 2015, and Physicians’ Desk Reference (71st ed. 2016) for information regarding therapies (e.g., prophylactic or therapeutic agents) which have been or are currently being used for the treatment of a disease state associated with inhibition of T-cell activation.

Non-limiting examples of one or more other therapies that can be used in combination with the invention include immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan.

### Urolithins

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

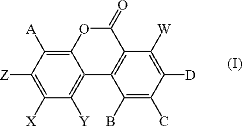

Substituent of structure (I)

| | | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (I) may be used in the embodiments of the invention.

In one embodiment of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the embodiments of the present invention is urolithin A, urolithin B, urolithin C or urolithin D. Most preferably, the urolithin used is urolithin A.

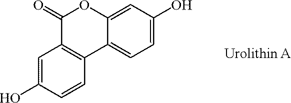 [SMILES: O=c1oc2cc(O)ccc2c2ccc(O)cc12]

According to one embodiment there is provided a combination of the invention wherein the compound of formula (I) is urolithin A.

According to one embodiment there is provided a combination of the invention wherein the compound of formula (I) is urolithin B.

According to one embodiment there is provided a combination of the invention wherein the compound of formula (I) is urolithin C.

According to one embodiment there is provided a combination of the invention wherein the compound of formula (I) is urolithin D.

In one embodiment, urolithins do not include acylated urolithins or optionally substituted acylated urolithins, (for example, acylated urolithin A, acylated urolithin B, acylated urolithin C, acylated urolithin D, acylated urolithin E, or acylated urolithin M5; orurolithin C having at least one hydroxyl substituted with a group containing a fatty acid). The term “acyl,” as used herein, represents a chemical substituent of formula -C(0)-R, where R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Examples of acyl include fatty acid acyls (e.g., short chain fatty acid acyls (e.g., acetyl)) and benzoyl.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D- glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl- propylamine, or a mono-,di- or trihydroxy lower alkylamine, for example mono-, di- or triethanolamine.

In a further embodiment, the urolithin is administered at a concentration of between 10 µM and 200 µM, for example between 10 µM and 150 µM, for example between 10 µM and 100 µM, for example between 10 µM and 80 µM, for example between 20 µM and 60 µM, for example between 40 µM and 60 µM, for example between 20 µM and 30 µM.

### Immunotherapy Treatment

Immunotherapy treatment for use in combinations of the invention include any treatment whose mechanism of action, in part or predominantly, acts via enhancing an individual’s immune response. For example, immune checkpoint blockage therapy, such as an anti-PD-1 antibody, an anti-PD-L1 antibody, an anti-CTLA4 antibody or a fusion protein, neoadjuvant immunotherapy and CAR-T immunotherapy (chimeric antigen receptor T-cell therapy),

### Anti-PD-1 Antibodies

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-⅟PD-L1 interaction mediates potent antitumour activity in preclinical models.

Human monoclonal antibodies (HuMAbs) that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics:
(a) binds to human PD-1 with a KD of 1 x 10⁻⁷ M or less, as determined by surface plasmon resonance using a Biacore biosensor system;
(b) does not substantially bind to human CD28, CTLA-4 or ICOS;
(c) increases T-cell proliferation in a Mixed Lymphocyte Reaction (MLR) assay;
(d) increases interferon-γ production in an MLR assay;
(e) increases IL-2 secretion in an MLR assay;
(f) binds to human PD-1 and cynomolgus monkey PD-1;
(g) inhibits the binding of PD-L1 and/or PD-L2 to PD-1;
(h) stimulates antigen-specific memory responses;
(i) stimulates antibody responses; and
(j) inhibits tumour cell growth in vivo.

Anti-PD-1 antibodies useful for combinations of the present invention include mAbs that bind specifically to human PD-1 and exhibit at least one, at least two, at least three, at least four, or at least five, of the preceding characteristics. In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumour T-cell functions (U.S. Pat. No. 8,008,449; Wang et al., Cancer Immunol Res. 2(9):846-56 (2014)). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody or fragment thereof cross-competes with pembrolizumab. In some embodiments, the anti-PD-1 antibody or fragment thereof binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also http://www.cancer.gov/drugdictionary?cdrid=695789 (last accessed: Jul. 29, 2019). Pembrolizumab has been approved by the FDA for the treatment of relapsed or refractory melanoma. In other embodiments, the anti-PD-1 antibody or fragment thereof cross-competes with MEDI0608. In still other embodiments, the anti-PD-1 antibody or fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089B2 or in https://www.cancer.gov/publications/dictionaries/cancer-drug/def/anti-pd-1-monoclonal-antibody-medi0680 (last accessed Jul. 29, 2019).

In certain embodiments, the first antibody is an anti-PD-1 antagonist. One example of the anti-PD-1 antagonist is AMP-224, which is a B7-DC Fc fusion protein. AMP-224 is discussed in U.S. Publ. No. 2013/0017199 or in http://www.cancer.gov/publications/dictionaries/cancer-drug?cdrid=700595 (last accessed Jul. 29, 2019).

In other embodiments, the anti-PD-1 antibody or fragment thereof cross-competes with BGB-A317. In some embodiments, the anti-PD-1 antibody or fragment thereof binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

In some embodiments, the antibody is Pidilizumab (CT-011), which is an antibody previously reported to bind to PD-1 but which is believed to bind to a different target. Pidilizumab is described in US Pat. No. 8,686,119B2 or WO 2013/014668 A1.

Anti-PD-1 antibodies useful for combinations of the present invention also include isolated antibodies that bind specifically to human PD-1 and cross-compete for binding to human PD-1 with nivolumab (see, e.g., U.S. Pat. Nos. 8,008,449 and 8,779,105; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g. WO 2013/173223).

In certain embodiments, the antibodies that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art. Anti-PD-1 antibodies useful for the compositions of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include:
(i) a Fab fragment, a monovalent fragment consisting of the V_{L}, V_{H}, C_{L}, and C_{H1} domains;
(ii) a F(ab′)2 fragment, a bivalent fragment comprising two Fab fragments linked by a disulfide bridge at the hinge region;
(iii) a Fd fragment consisting of the V_{H} and C_{H1} domains; and
(iv) Fv fragment consisting of the V_{L}, and V_{H} domains of a single arm of an antibody.

Anti-PD-1 antibodies suitable for use in the disclosed compositions are antibodies that bind to PD-1 with high specificity and affinity, block the binding of PD-L1 and/or PD-L2, and inhibit the immunosuppressive effect of the PD-1 signalling pathway. In any of the compositions or methods disclosed herein, an anti-PD-1 antibody includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgGI, IgG2, IgG3 or IgG4 isotype can be used. In certain embodiments, the anti-PD-1 antibody or antigen-binding portion thereof comprises a heavy chain constant region which is of a human IgGI or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al. Cancer Immunol Res. 2(9):846-56 (2014)). In yet other embodiments, the antibody comprises a light chain constant region which is a human kappa or lambda constant region. In other embodiments, the anti-PD-1 antibody or antigen-binding portion thereof is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, orBGB-A317. Because anti-PD-1 and anti-PD-L1 target the same signaling pathway and have been shown in clinical trials to exhibit similar levels of efficacy in a variety of cancers, including renal cell carcinoma (RCC) (see Brahmer et al. (2012) N Engl J Med 366:2455-65; Topalian et al. (2012a) NEngl J Med 366:2443-54; WO 2013/173223), an anti-PD-L1 antibody may be substituted for the anti-PD-1 Ab in any of the therapeutic methods disclosed herein. In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31 (suppl):3000. Abstract; U.S. Pat. No. 8,217,149) or MEDI4736 (Khieif (2013) In: Proceedings from the European Cancer Congress 2013; September 27-Oct. 1, 2013; Amsterdam, The Netherlands. Abstract 802). In certain embodiments, the antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

### Anti-PD-L1 Antibodies

In certain embodiments, the present application encompasses use of an anti-PD-L1 antibody in lieu of anti-PD-1 antibody. In one embodiment, the anti-PD-L1 antibody inhibits the binding of PD-L1 receptor, i.e., PD-1, with its ligand PD-L1. Anti-PD-L1 antibodies useful for the invention include antibodies engineered starting from antibodies having one or more of the V_{H} and/or V_{L}, sequences disclosed herein, which engineered antibodies can have altered properties from the starting antibodies. An anti-PD-L1 antibody can be engineered by a variety of modifications as described above for the engineering of modified anti-PD-1 antibodies of the invention.

In some embodiments, an anti-PD-L1 antibody useful for the present methods includes mAb 28-8 set forth in International Patent Application number: WO 2016/176503. In other embodiments, an anti-PD-L1 antibody useful for a combination of the invention comprises mAbs 28-1, 28-12, 29-8 and 20-12 (as disclosed in International Patent Application number: WO 2016/176503) or an antigen-binding portion thereof, for example, including Fab, F(ab′)2Fd, Fv, and scFv, di-scFv or bi-scFv, and scFv-Fc fragments, diabodies, triabodies, tetrabodies, and isolated CDRs.

### Anti-CTLA-4 Antibodies

Anti-CTLA-4 antibodies of the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

HuMAbs that bind specifically to CTLA-4 with high affinity have been disclosed in U.S. Pat. Nos. 6,984,720 and 7,605,238. Other anti-PD-1 mAbs have been described in, for example, U.S. Pat. Nos. 5,977,318, 6,051,227, 6,682,736, and 7,034,121. The anti-PD-1 HuMAbs disclosed in U.S. Pat. No. Nos. 6,984,720 and 7,605,238 have been demonstrated to exhibit one or more of the following characteristics:
(a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis;
(b) a kinetic association constant (kₐ) of at least about 10³, about 10⁴, or about 10⁵ m⁻¹ s⁻¹;
(c) a kinetic disassociation constant (k_{d}) of at least about 10³, about 10⁴, or about 10⁵ m⁻¹ s⁻¹; and
(d) inhibits the binding of CTLA-4 to B7-1 (CD80) and B7-2 (CD86).

Anti-CTLA-4 antibodies useful for the present invention include mAbs that bind specifically to human CTLA-4 and exhibit at least one, at least two, or at least three of the preceding characteristics.

An exemplary clinical anti-CTLA-4 antibody is the human mAb 10D1 (now known as ipilimumab and marketed as YERVOY®) as disclosed in U.S. Pat. No. 6,984,720. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

Another anti-CTLA-4 antibody useful for the present methods is tremelimumab (also known as CP-675,206). Tremelimumab is human IgG2 monoclonal anti-CTLA-4 antibody. Tremelimumab is described in WO2012/122444, U.S. Publ. No. 2012/263677, or WO 2007/113648 A2.

Anti-CTLA-4 antibodies useful for the disclosed composition also include isolated antibodies that bind specifically to human CTLA-4 and cross-compete for binding to human CTLA-4 with ipilimumab or tremelimumab or bind to the same epitope region of human CTLA-4 as ipilimumab or tremelimumab. In certain embodiments, the antibodies that cross-compete for binding to human CTLA-4 with, or bind to the same epitope region of human CTLA-4 as does ipilimumab or tremelimumab, are antibodies comprising a heavy chain of the human IgG1 isotype. For administration to human subjects, these cross-competing antibodies are chimeric antibodies, or humanized or human antibodies. Useful anti-CTLA-4 antibodies also include antigen-binding portions of the above antibodies such as Fab, F(ab′)₂, Fd or Fv fragments.

### Urolithin Administration/Dosage Regimes

The combinations of the present disclosure involve oral administration of a urolithin of formula (I) or salt thereof to a subject. Furthermore, adoptive T-cell therapy methods of the invention may comprise administration of a urolithin for a period following the T-cell transfer. Such administration can be in a daily amount in the range of 1.7 to 6.0 mmol per day, for example, from 1.7 to 2.7 mmol per day, or from 2.8 to 6.0 mmol per day, for a period between 2 to 16 weeks prior to vaccination. As discussed below, administration of is preferred in the range 250 mg to 1000 mg urolithin A (which corresponds to about 1.1 to 4.4 mmol) results in a surprisingly good pharmacokinetic profile, compared with a much higher dosage of 2000 mg. In one embodiment the dose is 250 mg/day, in an alternative embodiment the dose is 500 mg/day and in another embodiment the dose is 1000 mg/day.

In a further embodiment, administration doses are selected from:
250 mg once or twice a day;
500 mg once or twice a day;
750 mg once or twice a day;
1000 mg once or twice a day;
1250 mg once or twice a day; or
1500 mg once or twice a day

The methods of the present disclosure involve daily administration of the compound of formula (I) or salt thereof, or of a composition containing the compound or salt. In some embodiments the compound or composition is administered once per day, i.e. the compound or composition is to be administered at least once per 24 hour period. In other embodiments the compound, or composition comprising the compound, is administered multiple times per day, for example twice per day, or three or four times per day. In such cases, the daily dosage is divided between those multiple doses. In one embodiment administration is once a day, in a second embodiment administration is twice a day, in a third embodiment administration is three times a day.

The methods of the present disclosure would usually require daily administration of the compound of formula (I) or salt thereof, or of a composition containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

The methods of the present disclosure require daily administration of an amount of compound of formula (I) or salt thereof, of from 0.7 mmol per day up to 2.7 mmol per day thereof or from 0.7 mmol twice per day up to 2.7 mmol twice a day. In some embodiments, the amount administered is in the range of from 2.0 to 2.5 mmol. In some embodiments, the amount administered is approximately, 1.1, 1.2, 1.3, 1.4. 1.5, 1.6 1.7, 1.8, 1.9, 2.0, 2.1, 2.2, 2.3, 2.4, 2.5, 2.6, or 2.7 mmol. In other embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the method involves administration of approximately 2.2 mmol per day or 2.2 mmol twice per day of the compound of formula (I) or salt thereof (e.g. of urolithin A). The exact weight of compound that is administered depends on the molecular weight of the compound that is used. For example, urolithin A has a molecular weight of 228 g/mol (such that 2.20 mmol is 501.6 mg) and urolithin B has a molecular weight of 212 g/mol (such that 2.20 mmol is 466.4 mg).

In a further embodiment, the methods of the present disclosure require daily administration of an amount of compound of formula (I) or salt thereof, of from 2.8 mmol per day up to 6.0 mmol per day or twice per day thereof. In some embodiments, the amount administered is in the range of from 4.0 to 4.8 mmol. In some embodiments, the amount administered is approximately, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 mmol. In some preferred embodiments the method involves administration of approximately 4.4 mmol per day or twice per day of the compound of formula (I) or salt thereof (e.g. of urolithin A). The exact weight of compound that is administered depends on the molecular weight of the compound that is used. For example, urolithin A has a molecular weight of 228 g/mol (such that 4.40 mmol is 1003.2 mg) and urolithin B has a molecular weight of 212 g/mol (such that 4.40 mmol is 932.8 mg).

In some embodiments the methods involve administration of urolithin A in an amount in the range of from 400 to 600 mg/day or 400 to 600 mg twice per day. In a preferred embodiment the method involves administration of urolithin A in an amount in the range of from 450 to 550 mg, more preferably approximately 500 mg per day or twice per day.

In other embodiments the methods involve administration of urolithin A in an amount in the range of from 700 to 1300 mg/day twice per day, or in the range of from 750 to 1250 mg, or in the range of from 800 to 1200 mg, or in the range of from 850 to 1150 mg, or in the range of from 900 to 1100 mg per day or twice per day. In a preferred embodiment the method involves administration of urolithin A in an amount in the range of from 950 to 1150 mg/day or twice per day, more preferably approximately 1000 mg/day or twice per day.

In some preferred embodiments, the methods involve administering urolithin A to the subject in an amount in the range of from 4.5 to 11 mg/kg/day, such as 4.5 to 8.5 mg/kg/day. In another embodiment, the methods involve administering urolithin A to the subject in an amount in the range of 5 to 9 mg/kg/day. In another embodiment, the methods involve administering urolithin A to the subject in an amount in the range of from 6.0 to 8 mg/kg/day.

In other preferred embodiments, the methods involve administering urolithin A to the subject in an amount in the range of from 9 to 18 mg/kg/day such as 9 to 17 mg/kg/day. In another embodiment, the methods involve administering urolithin A to the subject in an amount in the range of from 10 to 17 mg/kg/day. In another embodiment, the methods involve administering urolithin A to the subject in an amount in the range of from 11 to 16 mg/kg/day.

Dosage regimes which combine a 500 mg dose and a 1000 mg dose may be advantageous. For example, a twice daily dosage regime which combines a first dose of 1000 mg and a second dose several hours later of 500 mg. Said 500 mg dose may be 6-18 hours after the 1000 mg dose, for example 8-12 hours after the 1000 mg dose. For example, about 12 hours after the 1000 mg dose. Thus, according to a further aspect of the invention there is provided the treatment of a disease with a compound of Formula (I) which comprises a twice daily dosage regime comprising a first dose of 1000 mg, followed by a second dose of 500 mg wherein the two doses are separated by 6-18 hours.

The compound of formula (I) or salt thereof, or composition containing the compound of salt, may be administered at any suitable time, for example it may be administered in the morning after sleep or in the evening. In some embodiments it may be preferable for the method to be performed at approximately the same time(s) each day, for example within 15, 30, 60 or 120 minutes of a given time point.

### Immunotherapy Administration/Dosage Regimes

The appropriate dose of an immunotherapy treatment is chosen based on clinical indications by a treating physician. Such treatment may comprise small molecule compounds or macromolecules such as antibodies.

For example, antibodies or functional parts thereof are administered in therapeutically effective amounts. Generally, a therapeutically effective amount may vary with the subject’s age, condition, and sex, as well as the severity of the medical condition of the subject. A therapeutically effective amount of an antibody or functional part thereof ranges from about 0.001 to about 30 mg/kg body weight, from about 0.01 to about 25 mg/kg body weight, from about 0.1 to about 20 mg/kg body weight, or from about 1 to about 10 mg/kg. The dosage may be adjusted, as necessary, to suit observed effects of the treatment.

In certain embodiments, antibodies or functional parts thereof are administered antibodies is individually administered at a dosage of at least about 0.1, at least about 0.3, at least about 0.5, at least about 1, at least about 3, at least about 5, at least about 10 or at least about 20 mg/kg, e.g., at least about 1 to at least about 10 mg/kg, e.g, at least about 1 to at least about 3 mg/kg, e.g, at least about 3 mg/kg, e.g, at least about 1 mg/kg. Antibodies or functional parts thereof can be administered at a dosing frequency of at least about once every week, at least about once every 2 weeks, at least about once every 3 weeks, or at least about once every 4 weeks, or at least about once a month, for up to 6 to up to 72 doses, or for as long as clinical benefit is observed, or until unmanageable toxicity or disease progression occurs. In some embodiments, antibodies or functional parts thereof are administered at a dosage of about 1 or about 3 mg/kg. In certain embodiments, the sequenced regimen comprises administering the antibodies or functional parts thereof antibody to the subject at a dosing frequency of once about every week, once about every 2 weeks, once about every 3 weeks, or once about every 4 weeks, or once a month for 6 to 72 doses, or for as long as clinical benefit is observed, or until unmanageable toxicity or disease progression occurs. In other embodiments, the antibodies or functional parts thereof are administered at a dosage of about 1 mg/kg at a dosing frequency of once about every 3 weeks for up to 48 doses.

In one embodiment, the compound of formula (I) is administered daily and the immunotherapy treatment, for example an antibody, is administered every 1 to 4 weeks, such as every 2 to 4 weeks, for example, every 2 or 3 weeks. In general, treatment duration is up to months remission.

Antibodies may be given as a bolus dose, to maximize the circulating levels of antibodies for the greatest length of time after the dose. Continuous infusion may also be used after the bolus dose.

### Compositions

The methods of the present disclosure preferably involve oral administration of the compound of formula (I) or a salt thereof. Any suitable oral composition containing the compound of formula (I) or salt thereof may be used. Accordingly, the use of a range of compositions which contain the compound of formula (I), and which are suitable for oral administration, is envisaged. Thus in some embodiments, the compound of formula (I), or salt thereof, is administered in the form of an oral composition containing the compound of formula (I) or salt thereof and one or more excipients suitable for oral administration. Oral compositions may comprise compositions having the form of a pill, tablet, capsule, caplet, lozenge, pastille, granules, powder for suspension, oral solution, oral suspension, oral emulsion, syrup, or the like.

In a further embodiment of the invention, a compound of formula (I) is administered by any means known to the skilled person for administration of pharmaceutical such as, intramuscular, sublingual, cutaneous, inhalation, ocular and auricular.

Compositions containing the compound of formula (I) may take any physical form suitable for the intended application, for example, they may be in the form of a solid (for example, a tablet or capsule), a semi-solid (for example, a softgel), or a liquid (including emulsions). In some instances, the composition may be in the form of a viscous fluid or a paste. Semi-solid forms may likewise contain excipients conventional in the art. The excipients can, for example, provide a desired hardness, shelf-life and flavour such that the composition has an acceptable taste, an attractive appearance and good storage stability. Semi-solid forms can be in the form of a paste. Where the composition is a softgel, it may for example be provided in a capsule having a shell. The shell may be of a conventional type, for example it may be a soft gelatin-based shell. By way of example, the composition may also be provided inside a hard capsule type of shell. Liquid compositions may be in the form of a medicine, a dietary supplement, or a beverage, each for oral consumption. Liquid formulations may be solutions, emulsions, slurries or other semi-liquids. Excipients in a liquid composition can, for example, provide a shelf-life, visual appearance, flavour and mouth-feel such that the composition has an acceptable taste, an attractive appearance and good storage stability. At certain levels of dilution, a drink may need to be shaken before the subject drinks it, so as to maintain an even suspension of the active ingredient.

In some preferred embodiments, the method comprises administration of a compound of formula (I) or salt thereof (e.g. urolithin A), in micronized form. Micronization enables the compound of formula (I) to disperse or dissolve more rapidly. Micronisation can be achieved by methods established in the art, for example compressive force milling, hamermilling, universal or pin milling, or jet milling (for example spiral jet milling or fluidised-bed jet milling) may be used. Jet milling is especially suitable. If micronized compound is used, then preferably the compound has a D₅₀ size of under 100 µm - that is to say that 50% of the compound by mass has a particle diameter size of under 100 µm. More preferably, the compound has a D₅₀ size of under 75 µm, for example under 50 µm, for example under 25 µm, for example under 20 µm, for example under 10 µm. More preferably, the compound has a D₅₀ in the range 0.5-50 µm, for example 0.5 to 20 µm, for example 0.5 to 10 µm, for example 1.0 to 10 µm, for example 1.5 to 7.5 µm, for example 2.8 to 5.5 µm. Preferably, the compound has a D₉₀ size of under 100 µm. More preferably, the compound has a D₉₀ size of under 75 µm, for example under 50 µm, for example under 25 µm, for example under 20 µm, for example under 15 µm. The compound preferably has a D₉₀ in the range 5 to 100 µm, for example 5 to 50 µm, for example 5 to 20 µm, for example 7.5 to 15 µm, for example 8.2 to 16.0 µm. Preferably, the compound has a D₁₀ in the range 0.5 - 1.0 µm. Preferably, the compound of formula (I) or salt thereof (e.g. urolithin A) has a D₉₀ in the range 8.2 to 16.0 µm, a D₅₀ in the range 2.8 to 5.5 µm and a D₁₀ in the range 0.5 to 1.0 µm.

In a further embodiment, the compound of formula (I) or salt thereof has a size distribution selected from one of the following:
(i) D₅₀ size in the range 0.5 to 50 µm and a D₉₀ size in the range 5 to 100 µm,
(ii) the compound has a D₉₀ size in the range 8.2 to 16.0 µm, a D₅₀ size in the range 2.8 to 5.5 µm and a D₁₀ size in the range 0.5 to 1.0 µm;
(iii) the compound of Formula (I) has a D₅₀ size in the range 0.5 to 20 µm and a D₉₀ size in the range 5 to 50 µm;
(iv) the compound of Formula (I) has a D₅₀ size under 50 µm and a D₉₀ size under 75 µm;
(v) the compound of Formula (I) has a D₅₀ size under 25 µm and a D₉₀ size under 50 µm;
(iv) the compound of Formula (I) has a D₅₀ size under 10 µm and a D₉₀ size under 20 µm;
(v) the compound of Formula (I) has a D₅₀ size under 10 µm and a D₉₀ size under 15 µm; or
(vi) the compound of Formula (I) has a D₅₀ size of 10 µm and a D₉₀ size of 20 µm.

### Compositions Comprising a Urolithin or Salt Thereof, and a Medium Chain Triglyceride

In some preferred embodiments, the compound of formula (I) or salt thereof (e.g. urolithin A) is administered in the form of a composition comprising: a) a medium-chain triglyceride; and b) the compound of formula (I) or salt thereof. Within those embodiments, preferably the compound of formula (I) (e.g. urolithin A) is in micronized form.

By selecting suitable medium chain triglycerides and excipients, the physical form of the composition can be tailored to the requirements of the product in question. For example, in some embodiments the compositions may be pharmaceutical compositions. In some embodiments the compositions may be nutritional compositions.

In many cases, compositions containing a compound of formula (I) and a medium chain triglyceride have the consistency of a viscous liquid or paste, and can be provided as a single serving supplement to a subject’s general diet (for example in a bar, gel, or a softgel capsule, hard capsule, or diluted in a drink); alternatively, it can be provided as a part of or the whole of a meal.

Where the methods of the disclosure involve use of a composition comprising a medium-chain triglyceride, the medium-chain triglyceride typically makes up at least 1% w/w of the composition, for example at least 5% w/w, for example at least 10% w/w, for example at least 15% w/w. The medium-chain triglyceride preferably makes up 20% w/w or more of the composition, for example 25% w/w or more by weight, for example 30% w/w or more by weight of the composition. For example the medium-chain triglyceride may make up 1-40% w/w of the composition, 2-40% w/w of the composition, 5-40% w/w of the composition; 10-40% w/w of the composition; 1-99% w/w of the composition, 5-99% w/w of the composition, 10-99% w/w of the composition, 20-99% w/w of the composition, 5-90% w/w of the composition, 10-90% w/w of the composition, for example 20-90% w/w of the composition, 20-80% w/w of the composition for example, 30-80% w/w of the composition, for example 30-70% w/w of the composition, for example 30-60% w/w of the composition, for example 30-50% w/w of the composition, for example 30-40% w/w of the composition, for example 30-35% w/w of the composition. For example the medium-chain triglyceride may make up 40-70% w/w of the composition, for example 50-70% w/w of the composition, for example, 55-65% w/w of the composition.

In such compositions, the compound of formula (l) typically makes up from 0.1 to 80% w/w of the composition, for example 0.1 to 60% w/w, for example 0.25 to 50% w/w. For example the compound of formula (l) may make up 0.5-50% w/w of the composition. If the composition is provided as a part or the whole of a meal then the compound of formula (I) may for example make up 0.25-5% w/w of the composition, for example, 0.3-3% w/w of the composition. If the composition is provided as a single serving supplement to a subject’s general diet, then the urolithin typically makes up from 20 to 80% w/w of the composition, for example 20 to 40% w/w, for example 25 to 35% w/w of the composition. For example the urolithin may make up 26-34% w/w of the composition, for example, 28-33% w/w of the composition; for example, 29-32% w/w of the composition, for example 29-31 % w/w of the composition.

In such compositions, the weight ratio of the medium-chain triglyceride component to the compound of formula (l) is generally in the range 0.01:1 to 100:1, for example 0.5:1 to 100:1, for example 0.5:1 to 50:1, for example 0.5:1 to 5:1; or, for example, 1:1 to 75:1, for example 1:1 to 50:1, for example 1:1 to 20:1, for example 1:1 to 10:1, for example 1:1 to 2.5:1, for example 1:1 to 2:1, for example 1:1 to 1.5:1. The weight ratio may be in the ratio 0.01:1 to 10:1, for example 0.1:1 to 10:1 or 0.01:1 to 5:1, for example 0.01:1 to 0.1:1.

In some preferred embodiments, the method of the present disclosure involves administration of a softgel capsule comprising a filling, which filling comprises the compound of formula (l) or salt thereof (e.g. urolithin A) and one or more medium-chain triglycerides. Within those embodiments, preferably the compound of formula (l) or salt thereof (e.g. urolithin A) is micronized. In embodiments where a softgel capsule is used, the shell component may be produced using conventional ingredients.

Medium-chain triglycerides are compounds of formula CH₂(OR¹)-CH(OR²)-CH₂(OR³) where R¹, R² and R³ are medium chain fatty acid groups, generally of formula -C(=O)(CH₂)ₙCH₃ where n is in the range 4 to 10, for example 6 to 8. Medium-chain fatty acids are fatty acids which have an aliphatic tail of 6 -12 carbon atoms. The aliphatic tail is predominantly saturated. Particular medium-chain fatty acids include caproic acid (hexanoic acid, C6:0), caprylic acid (octanoic acid, C8:0), capric acid (decanoic acid, C10:0) and lauric acid (dodecanoic acid, C12:0). Myristic acid (tetradecanoic acid, C14:0) can also be present in minor amounts. Medium-chain triglycerides most commonly used generally have a mixture of triglycerides of caprylic acid and capric acid, and contain 95% or greater of saturated fatty acids. The medium chain triglyceride component present in preferred compositions used in the methods of the present disclosure may consist of a homogeneous, single medium chain triglyceride compound type; more commonly, the medium chain triglyceride component is a mixture of two or more different medium chain triglyceride compounds.

The European Pharmacopoeia describes medium-chain triglycerides as the fixed oil extracted from the hard, dried fraction of the endosperm of Cocos nucifera L. (coconut) or from the dried endosperm of Elaeis guineenis Jacq. (African oil palm). The European Pharmacopoeia and the USPNF both have specifications for medium-chain triglycerides that require the presence of particular fatty acids is as follows: caproic acid (C6) ≤2.0%; caprylic acid(C8) 50.0-80.0%; capric acid (C10) 20.0-50.0%; lauric acid (C12) ≤3.0%; and myristic acid (C14) ≤1%.

Medium-chain triglycerides for use in preferred compositions comprise a mixture of triglycerides with fatty acid chains present in the following proportions: C6 ≤5%; C8 50-70%; C10 30-50%; and C12 ≤12%, for example C6 ≤0.5%; C8 55-65%; C10 35-45%; and C12 ≤1.5%.

Medium-chain triglycerides used in the preferred compositions may be derived from any known or otherwise suitable source.

Compositions used in the methods of the present disclosure may, advantageously, comprise one or more phospholipids. A particularly preferred phospholipid is phosphatidylcholine. The advantages brought about by phosphatidylcholine may be due, at least in part, to their amphipathic nature, e.g. due to properties as an emulsifier.

A particularly useful source of phospholipids, in particular phosphatidylcholine, is lecithin, and compositions used in the methods of the present disclosure advantageously comprise lecithin. Lecithin, when present in compositions, typically makes up at least 0.5% w/w of the composition, preferably at least 1% w/w of the composition. The lecithin preferably makes up 10% w/w or more of the composition, for example 20% w/w or more by weight, for example 30% w/w or more by weight of the composition. For example the lecithin may make up 0.5-80% w/w of the composition, for example 1-80% w/w, for example 20-80% w/w, for example 40-80% w/w, alternatively for example 0.5-75% w/w of the composition, for example, 1-40% w/w of the composition, for example 30-40% w/w of the composition, for example 30-35% w/w of the composition, for example, 30-75% w/w of the composition. Alternatively, the lecithin may make up 0.5-5% w/w of the composition, for example 1-5% w/w of the composition, for example 1-3% w/w of the composition, for example, 0.5-2% w/w of the composition, for example, 1-2% w/w of the composition. The weight ratio between the lecithin, when present, and the urolithin is generally in the range 0.02:1 to 3:1, for example, 0.03:1 to 1.2:1, for example 1:1 to 1.2:1, for example 1.1:1 to 1.2:1.

Commercially produced lecithin, which may be used in compositions described herein, typically contains the following major components: 33-35% soybean oil, 20-21 % inositol phosphatides, 19-21 % phosphatidylcholine, 8-20% phosphatidylethanolamine, 5-11 % other phosphatides, 5% free carbohydrates, 2-5% sterols and 1% moisture.

Commercially produced lecithin, which may be used in compositions described herein, may for example be enriched with phosphatidylcholine, having a minimum of 5% w/w phosphatidylcholine in the lecithin, for example, having a minimum of 10% w/w phosphatidylcholine in the lecithin, for example, having a minimum of 15% w/w phosphatidylcholine in the lecithin, for example, having a minimum of 20% w/w phosphatidylcholine in the lecithin, for example, having a minimum of 25% w/w phosphatidylcholine in the lecithin, for example, having a minimum of 30% w/w phosphatidylcholine in the lecithin, for example, having a minimum of 32% w/w phosphatidylcholine in the lecithin, for example, having a minimum of 40% w/w phosphatidylcholine in the lecithin.

Lecithins may also be modified by one or more of the following processes to tailor their properties: alcohol extraction of particular phospholipids to produce a lecithin with a modified ratio of differing phospholipids; acetone extraction to remove oil, resulting in a powdered or granulated phospholipid blend; spray drying onto proteins as carriers; spray cooling with synthetic emulsifiers such as high melting mono- and di-glycerides to produce flaked or powdered products; modification by enzyme action (phospholipases, commonly in particular phospholipase A2), in particular partial hydrolysis to produce lecithins with pronounced emulsifying behaviour; hydrolysis of fatty acid groups by acids and alkali; acetylation; and hydroxylation of fatty acid chains and amino groups.

In some embodiments, the methods comprise administration of a composition comprising a compound of formula (l) or salt thereof, a medium chain triglyceride, and an emulsifier (e.g. lecithin).

Pharmaceutical compositions containing the compound of formula (l) or salt thereof may for example include additional pharmaceutically active compounds.

Additional components in a composition may be compounds that do not provide health benefits to the subject, but instead improve the composition in some other way, for example its taste, texture or shelf-life as mentioned above. The composition may thus further contain one or more compounds selected from emulsifiers, colorants, preservatives, gums, setting agents, thickeners, sweeteners and flavourings.

Suitable emulsifiers, stabilisers, colorants, preservatives, gums, setting agents and thickeners are well known in the art of manufacture of emulsions and other semi-liquids. Emulsifiers may include one or more of phosphatidylcholine, lecithin, polysorbates such as polysorbate 60 or polysorbate 80 (Tween-60 and Tween-80), and glycerol monostearate (GMS). Glycerol monostearate is also known as glyceryl monostearate.

Stabilisers may be used in a composition described herein. Many compositions are stable suspensions without the need for an added stabiliser. A stable suspension is one that does not undergo a phase separation over time. For certain compositions, the stability can be improved by inclusion of an added stabiliser. Suitable stabilisers for use in compositions of the invention include glycerol monostearate (GMS), silicon dioxide and vegetable shortening. An exemplary stabiliser is GMS and preferred compositions of the invention contain GMS. Its properties also make GMS a good solvent for phospholipids, such as found in lecithin for example. GMS exists in two polymorphs: the α-form is dispersible and foamy, useful as an emulsifying agent or preservative. The β-form is suitable for wax matrices. The α-form is converted to the β-form when heated at 50° C.

GMS falls into two distinct grades: 40-55 percent monoglycerides, and 90 percent monoglycerides. 40-55 percent monoglycerides as defined by the European Pharmacopoeia describes GMS as a mixture of monoacylglycerols, mostly monostearoylglycerol, together with a quantity of di- and tri-glycerols. In particular, the 40-55 grade contains 40-55% monoacylglycerols, 30-45% diacylglycerols, and 5-15% of triacylglycerols. The 99 percent grade contains not less than 90% of monoglycerides. The monoglycerides in commercial GMS products are mixtures of variable proportions of glyceryl monostearate and glyceryl monopalmitate. The European Pharmacopoeia further divides glyceryl monostearate 40-55 into three types according to the proportion of stearic ester in the mixture. Type 1 contains 40.0-60.0% stearic acid, and the sum of palmitic and stearic acids is 90%. Type 2 contains 60.0-80.0% stearic acid, and the sum of palmitic and stearic acids is ≤90%. Type 3 contains 90.0-99.0% stearic acid, and the sum of palmitic and stearic acids is ≤96%. Any form of GMS may be used in the compositions.

In some embodiments, the method comprises administration of a composition comprising a medium chain triglyceride, the compound of formula (l) or a salt thereof (e.g.urolithin A), and a stabiliser, for example glycerol monostearate. In some embodiments the method involves administration of a composition comprising an emulsifier and a stabiliser.

Metal chelators or sequestrants such as sodium calcium salts of ethylenediamine tetra acetic acid (EDTA) may also be used. Other components that may be included in formulations of the invention include polyethylene glycols, silicon dioxide, vegetable shortening and beeswax.

A flavouring may be beneficial in compositions used in the methods described herein. In a liquid or semi-liquid composition, fruit flavour can be provided for example by inclusion of a fruit sauce or puree. Typical flavourings include strawberry, raspberry, blueberry, apricot, pomegranate, peach, pineapple, lemon, orange and apple. Generally, fruit flavourings include fruit extract, fruit preserve or fruit puree, with any of a combination of sweeteners, starch, stabilizer, natural and/or artificial flavours, colourings, preservatives, water and citric acid or other suitable acid to control the pH.

A unit dose composition used in the methods described herein preferably contains 250 mg or 500 mg of the compound of formula (l), for example 250 mg or 500 mg of urolithin A. A unit dose may for example be in the form of a tablet or capsule, in the form of a drink provided in a container such as a bottle or pouch sufficient to hold a single dose (e.g. 50 to 500 ml, 100 to 300 ml, for example, 250 ml or 500 ml). In a further alternative example, which is preferred, the unit dose is in the form of a softgel capsule, e.g. containing 250 mg of urolithin A.

A representative urolithin composition is shown in the Table below:

Representative composition A

| | |
|---|---|
| Composition | Per 100 g |
| Medium Chain Triglycerides | 10-85 g |
| Urolithin A | 10-50 g |
| Lecithin (comprising minimum phosphatidylcholine content of 32%w/w) | 10-50 g |
| Glycerol Monostearate | 0-5 g |

### Immunotherapy Treatment Compositions

Provided herein are compositions comprising an immunotherapy treatment, for example, an antibody or a fusion protein for use in a combination of the invention. Also provided herein are compositions comprising an anti-PD-1 antibody, an anti-PD-L1 antibody, an anti-CTLA4 antibody or a fusion protein. The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. The compositions (e.g. pharmaceutical compositions) comprise an effective amount of an immunotherapy treatment, such as an anti-PD-1 antibody, an anti-PD-L1 antibody, an anti-CTLA4 antibody or a fusion protein and a pharmaceutically acceptable carrier. In specific embodiments, the compositions (e.g., pharmaceutical compositions) comprise an effective amount of one or more antibodies or proteins, such as an anti-PD-1 antibody, an anti-PD-L1 antibody, an anti-CTLA4 antibody or a fusion protein.

Pharmaceutical compositions may be formulated in any conventional manner using one or more pharmaceutically acceptable carriers, such as adjuvants, or excipients.

Adjuvants include, but are not limited to, Freund’s adjuvant (complete and incomplete) or MF59C.1 adjuvant.

Suitable pharmaceutical carriers include sterile liquids, such as water and oils, including those of petroleum, animal, vegetable or synthetic origin, such as peanut oil, soybean oil, mineral oil, sesame oil and the like. In one embodiment, water is a carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

In a specific embodiment, an immunotherapy treatment for use in a combination of the invention is administered to a subject in accordance with the methods described herein and is administered as a pharmaceutical composition.

Generally, the components of the pharmaceutical compositions comprising an immunotherapy treatment, such as an anti-PD-1 antibody, an anti-PD-L1 antibody, an anti-CTLA4 antibody or a fusion protein are supplied either separately or mixed together in unit dosage form, for example, as a dry lyophilized powder or water free concentrate in a hermetically sealed container such as an ampoule or sachette indicating the quantity of active agent. Where the immunotherapy treatments, such as an anti-PD-1 antibody, an anti-PD-L1 antibody, an anti-CTLA4 antibody or a fusion protein is to be administered by infusion, it can be dispensed with an infusion bottle containing sterile pharmaceutical grade water or saline (e.g., PBS). Where the immunotherapy treatment, such as an anti-PD-1 antibody, an anti-PD-L1 antibody, an anti-CTLA4 antibody or a fusion protein, is administered by injection, an ampoule of sterile water for injection or saline can be provided so that the ingredients may be mixed prior to administration.

In some embodiments, the immunotherapy treatment, such as an anti-PD-1 antibody, an anti-PD-L1 antibody, an anti-CTLA4 antibody or a fusion protein, may be formulated for administration by any method known to one of skill in the art, including but not limited to, parenteral (e.g., subcutaneous, intravenous, intra-tumoural or intramuscular) administration. In one embodiment, the immunotherapy treatment, such as an anti-PD-1 antibody, an anti-PD-L1 antibody, an anti-CTLA4 antibody or a fusion protein, is formulated for local or systemic parenteral administration, for example intra-tumoural administration. In a specific embodiment, the immunotherapy treatment, such as an anti-PD-1 antibody, an anti-PD-L1 antibody, an anti-CTLA4 antibody or a fusion protein, is formulated for subcutaneous or intravenous administration, respectively. In one embodiment, the immunotherapy treatment, such as an anti-PD-1 antibody, an anti-PD-L1 antibody, an anti-CTLA4 antibody or a fusion protein, is formulated in a pharmaceutically compatible solution.

The immunotherapy treatment, such as an anti-PD-1 antibody, an anti-PD-L1 antibody, an anti-CTLA4 antibody or a fusion protein, can be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For the avoidance of doubt, combinations of the invention may be formulated in the same composition or formulated in separate compositions for simultaneous, separate or sequential administration.

Also within the scope of the present invention are kits, including pharmaceutical kits, comprising a combination of the invention for therapeutic uses. Kits typically include a label indicating the intended use of the contents of the kit and instructions for use. The term “label” includes any writing, or recorded material supplied on or with the kit, or which otherwise accompanies the kit. Certain embodiments of a pharmaceutical kit, comprise a urolithin, for example, urolithin A, and an immunotherapy treatment, such as an immune checkpoint blockage therapy in unit dosage form.

In one embodiment there is provided, a kit for the treatment of a disease state associated with inhibition of T-cell activation, comprising:
(a) a urolithin;
(b) an immunotherapy treatment, for example, an immune checkpoint blockage therapy;
(c) a container, or containers, for containing said agents; and
(d) optionally instructions for simultaneous, separate or sequential administration.

The term ‘about’ refers to a tolerance of ±20% of the relevant value, for example ±15% of the relevant value, such as ±10% of the relevant value or ±5% of the relevant value.

The term ‘adoptive T-cell transfer’ or ‘adoptive T-cell therapy’ refers to the transfer of T-cells into a patient. The cells may have originated from the patient or from another individual. The cells are administered with the goal of improving immune functionality and characteristics. The cells may be treated and/or expanded in number prior to administration to the patient.

The term ‘administer’ with respect to treating of cells refers to any method wherein cells come into contact with a compounds, such as a urolithin, for example by being injected or poured into a cell culture, or by the cells being added to a medium that contains the urolithin.

The term ‘anergy’ refers to any dysfunction observed in T cells characterized by lack of proliferation, cytokine production, or decrease in the phosphorylation of TCR signal intermediates following subsequent stimulation under conditions that would typically activate a naïve T cell.

The term ‘antibody or functional part thereof’ is used in the broadest sense. It may be man-made such as monoclonal antibodies (mAbs) produced by conventional hybridoma technology, recombinant technology and/or a functional fragment thereof. It may include both intact immunoglobulin molecules for example a polyclonal antibody, a monoclonal antibody (mAb), a monospecific antibody, a bispecific antibody, a polyspecific antibody, a human antibody, a humanized antibody, an animal antibody (e.g. camelid antibody), chimeric antibodies, as well as portions, fragments, regions, peptides and derivatives thereof (provided by any known technique, such as, but not limited to, enzymatic cleavage, peptide synthesis, or recombinant techniques), such as, for example, immunoglobulin devoid of light chains, Fab, Fab′, F (ab′)2, Fv, scFv, antibody fragment, diabody, Fd, CDR regions, or any portion or peptide sequence of the antibody that is capable of binding antigen or epitope. In one embodiment, the functional part is a single chain antibody, a single chain variable fragment (scFv), a Fab fragment, or a F(ab′)2 fragment.

An antibody or functional part is said to be “capable of binding” a molecule if it is capable of specifically reacting with the molecule to thereby bind the molecule to the antibody. Antibody fragments or portions may lack the Fc fragment of intact antibody, clear more rapidly from the circulation, and may have less non-specific tissue binding than an intact antibody. Examples of antibody may be produced from intact antibodies using methods well known in the art, for example by proteolytic cleavage with enzymes such as papain (to produce Fab fragments) or pepsin (to produce F(ab′)2 fragments). Portions of antibodies may be made by any of the above methods, or may be made by expressing a portion of the recombinant molecule. For example, the CDR region(s) of a recombinant antibody may be isolated and sub-cloned into an appropriate expression vector.

In one embodiment, an antibody or functional part is a human antibody. The use of human antibodies for human therapy may diminish the chance of side effects due to an immunological reaction in a human individual against nonhuman sequences. In another embodiment, the antibody or functional part is humanized. In another embodiment, an antibody or functional part is a chimeric antibody. This way, sequences of interest, such as for instance a binding site of interest, can be included into an antibody or functional part.

In one embodiment, the antibody may have an IgG, IgA, IgM, or IgE isotype. In one embodiment, the antibody is an IgG.

The term “autoimmune disease” refers to a subject’s disease characterized by cell, tissue and/or organ damage caused by the subject’s immune response to its own cells, tissues and/or organs. Exemplary autoimmune diseases include alopecia areata, ankylosing spondylitis, antiphospholipid syndrome, autoimmune Addison’s disease, autoimmune diseases of the adrenal glands, autoimmune hemolytic anemia, autoimmune hepatitis, autoimmune oophoritis, and Orchitis, autoimmune thrombocytopenia, Behcet syndrome, bullous pemphigoid, cardiomyopathy, stomatitis, diarrhea, dermatitis, chronic fatigue immune dysfunction syndrome, chronic inflammatory demyelinating polyneuropathy, colliculus Shier’s syndrome, scar pemphigoid, CREST syndrome, cold agglutinin disease, Crohn’s disease, discoid lupus, idiopathic mixed cryoglobulinemia, diabetes, eosinophilic muscle Meningitis, fibromyalgia-fibromyositis, glomerulonephritis, Graves’ disease, Guerrilla-Barr syndrome, Hashimoto’s thyroiditis, Hen-Scher’s purpura, idiopathic pulmonary fibrosis, idiopathic Sexual/autoimmune thrombocytopenic purpura, IgA neuropathy, juvenile arthritis, lichen planus, lupus erythematosus, Meniere syndrome, mixed connective tissue disease, multiple sclerosis, type 1 or immune-mediated diabetes, myasthenia gravis, Pemphigus-related diseases, pernicious anemia, polyarteritis nodosa, polychondritis, polyglandular syndrome, polymyalgia rheumatica, polymyositis, dermatomyositis, primary aglobulinemia, Primary biliary cirrhosis, psoriasis, psoriatic arthritis, Reye’s phenomenon, Reiter’s syndrome, rheumatoid arthritis, sarcoidosis, scleroderma, Sjogren’s syndrome, systemic myotonia syndrome, Systemic lupus erythematosus, Svet’s syndrome, Still’s disease, lupus erythematosus, Gaoan’s arteritis, transient arteritis/giant cell arteritis, ulcerative colitis, uveitis, vasculitis, vitiligo and Wegener Granulomatous disease.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukaemia, lung cancer and the like. The terms “tumour” and “cancer” are used interchangeably herein, e.g., both terms encompass solid and liquid, e.g., diffuse or circulating, tumours. As used herein, the term “cancer” or “tumour” includes premalignant, as well as malignant cancers and tumours.

The term “carrier” refers to a diluent, adjuvant, excipient, or vehicle with which the therapeutic is administered.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signalling domain (also referred to herein as “an intracellular signalling domain”) comprising a functional signalling domain derived from a stimulatory molecule and/or costimulatory molecule as defined below. In some embodiments, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signalling domain. In one aspect, the stimulatory molecule is the zeta chain associated with the T cell receptor complex. In one embodiment, the cytoplasmic signalling domain further comprises one or more functional signalling domains derived from at least one costimulatory molecule as defined below. In one aspect, the costimulatory molecule is chosen from the costimulatory molecules described herein, e.g., 4-1BB (i.e., CD137), CD27 and/or CD28. In one embodiment, the CAR comprises a chimeric fusion protein comprising an extracellular antigen binding domain, a transmembrane domain and an intracellular signalling domain comprising a functional signalling domain derived from a stimulatory molecule. In one embodiment, the CAR comprises a chimeric fusion protein comprising an extracellular antigen binding domain, a transmembrane domain and an intracellular signalling domain comprising a functional signalling domain derived from a costimulatory molecule and a functional signalling domain derived from a stimulatory molecule. In one embodiment, the CAR comprises a chimeric fusion protein comprising an extracellular antigen binding domain, a transmembrane domain and an intracellular signalling domain comprising two functional signalling domains derived from one or more costimulatory molecule(s) and a functional signalling domain derived from a stimulatory molecule. In one embodiment, the CAR comprises a chimeric fusion protein comprising an extracellular antigen binding domain, a transmembrane domain and an intracellular signalling domain comprising at least two functional signalling domains derived from one or more costimulatory molecule(s) and a functional signalling domain derived from a stimulatory molecule. In one embodiment, the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. For a review of CAR T Cell Immunotherapy, see Basher et al (2022) Pharmacology & Pharmacy, 13, 483-515.

The term ‘excipient’ refers to a substance formulated alongside the active ingredient of a medication, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity or enhancing solubility.

The abbreviation ‘HumAbs’ refers to humanized monoclonal antibodies.

The term ‘lecithin’ designates any group of fatty substances occurring in animal and plant tissues including phosphoric acid, choline, fatty acids, glycerol, glycolipids, triglycerides, and phospholipids (e.g., phosphatidylcholine, phosphatidylethanolamine, and phosphatidylinositol). Commercial lecithin obtained from soya and sunflower comprises the phospholipids phosphatidylcholine, phosphatidylinositol, phosphatidylethanolamine, and phosphatidic acid. Lecithin may be obtained by chemical extraction from its source in a nonpolar solvent such as hexane, ethanol, acetone, petroleum ether or benzene, or by mechanical extraction. In particular, lecithin may be obtained by extraction from sources including soybeans, eggs, milk, rapeseed, cottonseed and sunflower. Commercial lecithin for use in edible formulations may be readily purchased.

The term ‘immune checkpoint blockage therapy’ or ‘immune checkpoint blockage therapies’ relates to therapeutic approaches which remove inhibitory signals of T-cell activation which enable reactive T-cells to overcome regulatory mechanisms and mount an effective immune response, for example, tumour-reactive T cells to mount an effective anti-tumour response. For a review of checkpoint blockage therapy see: Wei et al(2018) Cancer Discovery 8(8), 1-18. Examples of immune checkpoints include: PD-1, CTLA-4, lymphocyte activation gene-3 (LAG-3), T-cell immunoglobulin and ITIM domain (TIGIT), and T-cell immunoglobulin-3 (TIM-3).

The term ‘immunotherapy treatment’ refers to any treatment whose mechanism of action, in part or predominantly, acts via enhancing an individual’s immune response.

The abbreviation ‘mAbs’ refers to monoclonal antibodies.

The term ‘PD-1 antagonist refers to any agent which blocks the inhibitory effect of PD-1 on the immune system. For example, a PD-1 antagonist includes agents, which directly block the binding of PD-1 to its receptor, and agents, which have an allosteric effect on the activity of PD-1.

The term “pharmaceutically acceptable” means approved by a regulatory agency of the Federal or a state government or listed in the U.S. Pharmacopeia or other generally recognized pharmacopeia for use in animals, and more particularly in humans.

The term “Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogues having at least one common epitope with hPD-1. The complete hPD-1 sequence can be found under GenBank Accession No. U64863.

The term “Programmed Death Ligand-1 (PD-L1)” is one of two cell surface glycoprotein ligands for PD-1 (the other being PD-L2) that down-regulate T cell activation and cytokine secretion upon binding to PD-1. The term “PD-L1” as used herein includes human PD-L1 (hPD-L1), variants, isoforms, and species homologs of hPD-L1, and analogues having at least one common epitope with hPD-L1. The complete hPD-L1 sequence can be found under GenBank Accession No. Q9NZQ7.

The term “Programmed Death Ligand-2 (PD-L2)” is one of two cell surface glycoprotein ligands for PD-1 (the other being PD-L2) that down-regulate T cell activation and cytokine secretion upon binding to PD-1. The term “PD-L2” as used herein includes human PD-L2 (hPD-L2), variants, isoforms, and species homologs of hPD-L2, and analogs having at least one common epitope with hPD-L2. The complete hPD-L1 sequence can be found under GenBank Accession No. Q9BQ51.

The term, “separate” administration means the administration of each of two or more compounds to a patient from non-fixed dose dosage forms simultaneously, substantially concurrently, or sequentially in any order. There may, or may not, be a specified time interval for administration of each the compounds.

The term “sequential” administration means the administration of each of two or more compounds to a patient from non-fixed (separate) dosage forms in separate actions. The administration actions may, or may not, be linked by a specified time interval. For example, administering compounds over a specified time such as once every 14 to 21 days..

The term “simultaneous” administration means the administration of each of two or more compounds to a patient in a single action such as where each compound are administered independently at substantially the same time or separately within time intervals that allow the compounds to show’ a cooperative therapeutic effect.

The term ‘T cell exhaustion’ refers to the state which occurs when T cells are chronically activated at sites of chronic inflammation, such as cancer, autoimmunity, and chronic infection’, leading to a loss of T-cell effector functions..

The term ‘T-cell population’ refers to a population of cells comprising T-cells, optionally comprising other cell types.

The ‘T-cell fitness’ refers to the ability of T-cells to perform their effector functions, such as antigen recognition, cytotoxicity and secretion of cytokines.

The invention will now be illustrated with respect to the following non-limiting examples.

### EXAMPLES

### Example 1: Urolithin A Suppresses Intestinal Tumor Growth in a T Cell Dependent Manner

To examine whether Urolithin A (UA)-dependent mitophagy mimicked the effect of Stat3^{DIEC} mice (Ziegler et al., 2018) and prevents intestinal tumor development in a T cell dependent manner, we employed a model of azoxymethane (AOM)-induced tumorigenesis. FVB mice were injected with the procarcinogen azoxymethane (AOM) once a week for six weeks and kept for 18 weeks on either a high-dose UA diet (2.28 g/kg), a low-dose UA diet (0.57 g/kg) or on control diet (FIG. 1A). While low-dose UA did not have an effect on tumor load, the high-dose containing diet led to a significant decrease in tumor incidence and tumor size (FIGS. 1B-D). Expectedly, this was accompanied by an increased infiltration of CD3⁺T cells (FIG. 1E). To confirm UA-induced mitophagy and to explore whether UA could be employed therapeutically in more advanced colorectal cancer, we took advantage of a recently developed tumor organoid system (Nicolas et al., 2022). We treated APTK organoids (characterized by loss of Apc, Trp53 and Tgfbr2 as well as expression of oncogenic Kras^{G12D}) for 48 hours with increasing concentrations of UA (FIG. 1F) and observed a dose-dependent formation of lysosomes (FIG. 1G) and a concomitant loss of MitoTracker staining (FIG. 1H). This was paralleled by a marked upregulation of MHC-I (FIG. 1I) validating our previous observation linking mitophagy in IEC to MHC-1 upregulation (Ziegler, 2018). To examine the therapeutic potential of UA, we subcutaneously (s.c.) transplanted APTK organoids into C57BL/6 mice (FIG. 1J). Tumor-bearing mice were subsequently subjected to the high-dose UA diet (2.28 g/kg), which resulted in significantly reduced tumor growth and enhanced CD8⁺ T cell infiltration (FIGS. 1K-M). When we transplanted APTK organoids s.c. into Rag1^{-/-} mice, the absence of mature T and B cells abrogated the protective effect of UA (FIG. 1N). Similarly, depletion of CD8⁺ T cells led to exacerbated tumor growth in both control and UA-fed cohorts (FIGS. 1O-P), confirming that UA-induced tumor suppression was T cell dependent. Consequently, we confirmed that UA sensitized APTK tumors to PD-1 blockade, while immune checkpoint inhibition alone did not affect APTK-tumor growth (FIGS. 1Q,R).

### Example 2: UA Promotes T_{SCM} Differentiation

Mitochondrial remodelling has been previously associated with alterations in T cell fate (Buck et al., 2016; Yu et al., 2020b). Given the observed dependency of the UA effect on CD8⁺ T cells, we wondered whether UA may also directly affect T cell fate commitment. Purified CD3+ T cells from FVB mice were stimulated with aCD3/aCD28 beads to induce T cell differentiation into effector T cell subsets in the presence or absence of UA for up to 72 hours (FIG. 2A). UA administration blocked differentiation into effector T cells (FIGS. 2B, C) and resulted in a significant increase of naïve CD44^{lo}CD62L^{hi} T cells (FIGS. 7A-7B). Previously, a rare naïve-like subset of T cells with enhanced stemness capabilities, termed T stem cell memory cells (T_{SCM}) has been identified (Gattinoni et al., 2009). T_{SCM} are marked by extreme longevity, their ability to self-renew, and potential for immune reconstitution (Gattinoni et al., 2017) which translates into potent anti-tumor immunity. Phenotypically, T_{SCM} represent a subset of minimally differentiated T cells, which share a CD44^{lo}CD62L^{hi} phenotype with naïve T cells, but are phenotypically distinct by expressing high levels of Sca1 (Gattinoni et al., 2009). Indeed, UA led in a dose dependent manner to a significantly increased number of these CD44⁻ CD62L+Sca1^{hi} T_{SCM} (FIG. 2D) that were characterized by a reduced mitochondrial membrane potential as well as increased CD95 expression (FIGS. 2E, F). UA restricted CD8+T cell division in a dose-dependent manner (FIGS. 7C-7D). This was associated with reduced cyclin D1 expression (FIG. 7E), in line with previous observations that link T_{SCM} reprogramming to halted proliferation (Gattinoni et al., 2009; Verma et al., 2021). Furthermore, we observed that T_{SCM} were only formed in activated T cells (FIG. 7F). Excluding the possibility that UA reduces TCR-mediated activation of T cells, thus limiting effector formation by retaining a naïve-like state, we found that UA treated T cells show equal phosphorylation of Stat1 (Gamero and Larner, 2000) compared to vehicle controls (FIG. 7G).

Also in vivo, UA administration led to a marked increase of CD8⁺CD44^{lo}CD62L^{hiSca1hi} T cells in APTK-induced tumors (FIG. 2G), yet no change in a antigen-presenting or immune-suppressing TME represented by dendritic cells (DCs), TAMs, or MDSC subsets (FIGS. 7H-7L). This indicates that Urolithin A directly acts on T cells, and their reduced exhaustion state is not a by-product of an altered TME in this model. Indeed, fitting the observation of self-dividing memory subsets (Kratchmarov et al., 2018; Utzschneider et al., 2016), we found increased expression of the transcription factor 1 (TCF1) in tumor infiltrating CD8⁺ T cells (FIG. 2H). Additionally, we observed a reduction of exhaustion markers such as PD-1, CTLA-4 and Tim3 in CD8⁺ T cells of UA-fed mice (FIGS. 2I-K) which allowed stronger induction of TNFa and IFNg when tumor infiltrating CD8⁺ T cells were restimulated with PMA/ionomycin ex vivo (FIGS. 2L,M). Collectively, this data indicates that UA treatment results in the formation of T_{SCM} in the TME, conferring superior CD8+-mediated antitumor immunity.

### Example 3: UA Improves Tumor Therapy by Adoptive T Cell Transfer

Adoptive cell transfer (ACT) represents the infusion of antigen-specific leukocytes with direct antitumor activity, yet identification, selection and expansion of lymphocyte subsets harboring optimal antitumor qualities remains one of the most crucial challenges (Rosenberg and Restifo, 2015). ACT benefits especially from minimally differentiated cells due to their improved survival and long-term potential to generate unexhausted effector cells (Luca Gattinoni et al; Mo et al., 2021; Roberto et al., 2015). In particular, CD8⁺ T cells restricted in a stem-like state have been associated with enhanced tumor suppressive properties upon adoptive cell transfer (Enrico Lugli et al; Verma et al., 2021). Having demonstrated that UA induces a T_{SCM} phenotype in murine T cells, we next investigated whether UA could be exploited to improve adoptive immunotherapy. We cultured CD3⁺ T cells from OT-1 donor mice (FIG. 3A) for 48 hours in the presence of UA or vehicle control, followed by adoptive transfer into immunodeficient Rag1^{-/-} mice. Seven days following the transfer the number of engrafted CD8⁺ T cells was significantly higher in mice that had received Urolithin A-treated T cells (FIG. 3B) supporting an increased potential to expand. Moreover, when UA-conditioned OT-1 T cells were transplanted into mice bearing palpable ovalbumin-overexpressing APTK (APTK-OVA) tumors (FIG. 3C), this led to a more substantial tumor suppression in comparison to control OT-I T cells (FIGS. 3D, E). Analysis of tumors revealed upon UA-T cell transfer a lower expression of CD44 and a higher number of TCF1^{Hi}CD8⁺ tumor infiltrating T cells (FIGS. 3F-G) in tumor in line with maintenance of a UA-induced memory phenotype (Schumann et al., 2015; Zhou et al., 2010). CD62L expression remained unchanged (FIG. 3H). Moreover, consistent with an improved memory response, animals receiving UA-pretreated T cells contained less Tim3^{hi}PD1^{hi} terminally exhausted CD8⁺ T cells in the TME (FIG. 3I). Thus, UA augments immune-mediated antitumor memory upon adoptive cell transfer.

### Example 4: Urolithin A Induces Pink1-Dependent Mitophagy in T Cells

Next we examined whether the shift towards CD44⁻CD62L⁺Sca1^{hi} T_{SCM} cells was triggered by UA-induced mitophagy in T cells. We confirmed a reduction of mitochondrial membrane potential within six hours after UA administration in CD8⁺ T cells (FIGS. 4A-B). This was accompanied by enhanced lysosome formation (FIG. 4C) and after 24 hours followed by the loss of mitochondrial content in a dose-dependent manner (FIG. 4D). The latter could be deteted in all T cell subsets analyzed (T_{SCM}, T_{CM} and T_{EFF}; FIG. 1E), thus suggesting induction of mitophagy.

UA activates mitophagy in myocytes and hippocampal neurons via a Pink1/Parkin mediated stress-response (D′Amico et al., 2021). Also in a-CD3/a-CD28 stimulated CD3⁺ T cells, UA led to a significant upregulation of autophagy genes and Pink1 (FIG. 4F). Moreover, UA stabilized Parkin protein expression (FIG. 4G). To functionally confirm the dependence of UA induced T_{SCM} formation on Pink1/Parkin mediated mitophagy, we activated Pink1^{-/-} CD3⁺ T cells in the presence or absence of UA. Loss of Pink1 prevented the UA-induced lysosome formation as well as the decrease in mitochondrial content (FIGS. 4H-I). Pink1^{-/-} CD8⁺ T cells failed to exhibit a T_{SCM} phenotype (FIG. 4J), and expressed less TCF-1 in C44⁻CD62L⁺CD8⁺ T cells (FIG. 4K). Consequently, Pink1 deletion abrogated the tumor suppressive effect of UA (FIG. 4L) while CD8⁺ TIL of Pink1^{-/-} mice did not exhibit differences in TCF-1 expression, exhaustion markers, or ex vivo cytokine release (FIGS. 4M-Q) strongly supporting the notion that UA-induced Pink1-dependent mitophagy triggers T_{SCM} formation to enhance antitumor immunity.

### Example 5: UA Induces T_{SCM} via Cytosolic Release of PGAM5 That Drives Wnt Signalling

To explore the downstream events linking mitophagy to T_{SCM} formation, we performed RNA sequencing to globally assess differential gene expression in T cells exposed for 48 hours to either DMSO or UA in vitro. We identified a total amount of 1178 differentially expressed genes of which 765 were significantly upregulated and 413 downregulated (FIG. 5A). UA treatment reduced the expression of genes that code for immune checkpoints and effector molecules, while enhancing expression of Cd27, Ccr7 and adhesion genes (FIG. 5B), a characteristic of stem-cell like CD8⁺ T cells (Enrico Lugli et al; Mo et al., 2021; Parisi et al., 2020; Reschke et al., 2021). Additionally, UA treated cells show special enrichment of genes involved in memory formation such as Tcf7, Bach2 and Bcl6 and reduced expression of effector fate associated genes Prdm1 and Id2 (Ichii et al., 2002; Roychoudhuri et al., 2016; Zhou et al., 2010) (FIG. 5C). In agreement with the increased number of TCF1^{hi} T_{SCM} cells, upstream regulator analysis of RNAseq data revealed Tcf7 as a possible regulator of UA-induced transcriptomic changes on T cells in vitro (FIG. 8A). Indeed, when we applied ICG001 to pharmacologically block TCF-1, UA-induced T_{SCM} formation in vitro was abrogated (FIGS. 5D-E).

Next we examined whether the observed upregulation of TCF1 was a result of enhanced Wnt signalling. In line with this notion, we observed an increased transcription of several Wnt target genes (FIG. 8B). Moreover, UA led to a marked decrease of b-catenin phosphorylation already after 6 hours (FIG. 5F) indicating activation of Wnt-signaling prior to the transcriptional changes. Mitophagy releases the mitochondrial-bound protein phosphatase phosphoglycerate mutase family member 5 (Pgam5) to the cytoplasm where it has been suggested to block axin-dependent b-catenin degradation thereby inducing mitochondrial biogenesis (Bernkopf et al., 2018; Yamaguchi et al., 2019). To examine whether Pgam5 may be involved in UA-induced Wnt activation, we examined Pgam5 localization upon UA administration. Immunoblot analysis of sub-cellular fractionations revealed that Pgam5 was indeed rapidly released into the cytoplasma upon UA-mediated mitophagy (FIG. 5G), which could also be confirmed by immunofluorescence (FIG. 5H). More importantly, loss of of Pgam5 blocked the UA-dependent expansion of CD44⁻CD62L⁺Sca1^{hi} T_{SCM} cells (FIG. 5I). UA treated Pgam5^{-/-} CD8⁺ T cells also failed to upregulate TCF1 or the memory marker CD95 in vitro (FIGS. 5J-K), despite the fact that UA still enhanced lysosome formation and reduced mitochondrial content (FIGS. 8C-8D) confirming that cytosolic Pgam5 contributed to UA-induced alterations of T cell function. In line with this notion, we confirmed lack of cytoplasmic expression of Pgam5 in Pink1^{-/-} CD8⁺ T cells upon UA exposure (FIG. 8E).

Pgam5 dependent Wnt activation has been suggested to trigger compensatory mitochondrial biogenesis in response to mitophagy (Bernkopf et al., 2018). Peroxisome proliferator-activated receptor gamma coactivator 1-alpha (PGC-1α) is considered the master regulator of mitochondrial biogenesis (Finck and Kelly, 2006). Accordingly, we detected a marked upregulation of PGC-1 after prolonged UA administration in vitro (FIG. 5L), which was accompanied by an increased mitochondrial content in TIL of APTK tumors (FIG. 5M) indicating mitochondrial biogenesis and in line with superior T cell fitness in the TME (Dumauthioz et al., 2020; Scharping et al., 2016). UA failed to enhance PGC-1α in Pink1^{-/-} or Pgam5^{-/-} CD8⁺ T cells (FIGS. 5N, O), while chemical inhibition of PGC-1α blocked UA-dependent expansion of CD44⁻CD62L⁺Sca1^{hi} T_{SCM} cells (FIG. 5R). Collectively, these results indicated that UA drives T_{SCM} formation via a Wnt-dependent upregulation of PGC-1a promoted by the cystosolic release of Pgam5 in response to mitophagy.

Finally, we aimed to determine whether UA causes expansion of T_{SCM} cells in human CD8⁺ T cells. We isolated human CD3⁺ T cells from PBMCs of healthy donors and stimulated them with aCD3/ aCD28 beads in vitro in the presence of UA (FIG. 5Q). Indeed, UA increased the frequency of human T_{SCM} cells based on the expression of CD45RA+CCR7^{hi}CD62L+CD95⁺CD8⁺ in five out of five individual donors (FIG. 5R, FIGS. 9A-9B). Like in murine T cells, after 48 hours UA-treated human CD8⁺ T cells displayed a decreased mitochondrial membrane potential (FIG. 5S) and intracellular staining confirmed increased TCF1 expression (FIG. 5T) confirming that UA induces memory stem cell features in both murine and human T cells.

To examine whether Pgam5 may be involved in UA-induced Wnt activation, we examined Pgam5 localization upon UA administration. Immunoblot analysis of sub-cellular fractionations revealed that Pgam5 was indeed rapidly released into the cytoplasma upon UA-mediated mitophagy (FIG. 5G), which could also be confirmed by immunofluorescence (FIG. 5H). More importantly, loss of of Pgam5 blocked the UA-dependent expansion of CD44⁻CD62L⁺Sca1^{hi} T_{SCM} cells (FIG. 5I). UA treated Pgam5^{-/-} CD8⁺ T cells also failed to upregulate TCF1 or the memory marker CD95 in vitro (FIGS. 5J-K), despite the fact that UA still enhanced lysosome formation and reduced mitochondrial content (FIGS. 8C-8D) confirming that cytosolic Pgam5 contributed to UA-induced alterations of T cell function. In line with this notion, we confirmed lack of cytoplasmic expression of Pgam5 in Pink1^{-/-} CD8⁺ T cells upon UA exposure (FIG. 8E).

Pgam5 dependent Wnt activation has been suggested to trigger compensatory mitochondrial biogenesis in response to mitophagy (Bernkopf et al., 2018). Peroxisome proliferator-activated receptor gamma coactivator 1-alpha (PGC-1α) is considered the master regulator of mitochondrial biogenesis (Finck and Kelly, 2006). Accordingly, we detected a marked upregulation of PGC-1 after prolonged UA administration in vitro (FIG. 5L), which was accompanied by an increased mitochondrial content in TIL of APTK tumors (FIG. 5M) indicating mitochondrial biogenesis and in line with superior T cell fitness in the TME (Dumauthioz et al., 2020; Scharping et al., 2016). UA failed to enhance PGC-1α in Pink1^{-/-} or Pgam5^{-/-} CD8⁺ T cells (FIGS. 5N, O), while chemical inhibition of PGC-1α blocked UA-dependent expansion of CD44⁻CD62L⁺Sca1^{hi} T_{SCM} cells (FIG. 5R). Collectively, these results indicated that UA drives T_{SCM} formation via a Wnt-dependent upregulation of PGC-1a promoted by the cystosolic release of Pgam5 in response to mitophagy.

Finally, we aimed to determine whether UA causes expansion of T_{SCM} cells in human CD8⁺ T cells. We isolated human CD3⁺ T cells from PBMCs of healthy donors and stimulated them with aCD3/ aCD28 beads in vitro in the presence of UA (FIG. 5Q). Indeed, UA increased the frequency of human T_{SCM} cells based on the expression of CD45RA+CCR7^{hi}CD62L+CD95⁺CD8⁺ in five out of five individual donors (FIG. 5R, FIGS. 9A-9B). Like in murine T cells, after 48 hours UA-treated human CD8⁺ T cells displayed a decreased mitochondrial membrane potential (FIG. 5S) and intracellular staining confirmed increased TCF1 expression (FIG. 5T) confirming that UA induces memory stem cell features in both murine and human T cells.

### Example 6: UA Promotes Human TSCM, Facilitating Generation of Potent CAR TSCM

Finally, we aimed to determine whether UA causes expansion of TSCM cells in human CD8+ T cells. We isolated human CD3+ T cells from PBMCs of healthy donors and stimulated them with □CD3/□CD28 beads in vitro in the presence of UA (FIG. 6A). Indeed, UA increased the frequency of human TSCM cells based on the expression of CD45RA+CCR7HiCD62L+CD95+CD8+ in five out of five individual donors (FIG. 6B, Supplementary FIGS. 5A, B). Like in murine T cells, after 48 hours UA-treated human CD8+ T cells displayed a decreased mitochondrial membrane potential (FIG. 6C) and intracellular staining confirmed increased TCF1 expression (FIG. 6D) validating that UA induces memory stem cell features in both murine and human T cells.

Infusion of T cells carrying an engineered chimeric antigen receptor (CAR), designed to specifically guide leukocytes to recognize and eliminate malignancies of interest, has shown impressive clinical results, but incomplete remissions and frequent relapses after successful therapy highlight the need to improve sustained antitumor response (June et al., 2018; Majzner and Mackall, 2019). Recent data suggests that CAR-T cells containing high fractions of TN/SCM show improved tumor killing and the exclusive ability to counteract leukemia re-challenge in hematopoietic stem/precursor cell-humanized mice (Arcangeli et al., 2022). To determine whether UA-triggered mitophagy also constitutes a feasible strategy to induce CAR-TSCM, activated T cells were transduced with the CD19-CAR gene by a lentiviral vector (VSV-LV) in the presence or absence of UA. Three days post transduction, the amount of CAR-expressing TSCM was determined (FIG. 6E; Supplemental FIG. 6A). UA did not impair gene delivery into CD8+ cells (Supplemental FIG. 6B), yet while CAR expressing TSCM cells were markedly increased and comprised about 60% of CD8+ cells upon UA exposure (FIG. 6F), this did not have a negative impact on CD19 CAR-T cell mediated killing of NALM-6 leukemia cells (FIG. 6G). Markers of exhaustion were not affected by UA supplementation (Supplemental FIG. 6C). Even when we applied UA to previously frozen CAR-T cells specific for carcinoembryonic antigen (CEA; FIG. 6H), this strongly enhanced CAR TSCM formation (FIG. 6I) with comparable killing efficacy of CEA-expressing human CRC organoids (FIG. 6J). Thus, UA markedly enhances expansion of CAR-TSCM cells

### Equivalents

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

### Incorporation by Reference

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

### Statements of Invention (I)

i) A urolithin for use in a method of overcoming or reversing of T-cell dysfunction.

ii) A urolithin for use as claimed in a) wherein the T-cell dysfunction is T-cell exhaustion.

iii) A urolithin for use as claimed in a) wherein the T-cell dysfunction is T-cell exclusion.

iv) A urolithin for use as claimed b) wherein the T-cell exhaustion is CD8+ T-cell exhaustion

v) A urolithin for use as claimed in a) wherein the T-cell dysfunction is anergy.

vi) A urolthin for use as claimed in e) wherein the anergy is CD8+ T-cell anergy.

vii) A urolithin for use as claimed in a) wherein the T-cell dysfunction is T-cell senescence.

viii) A urolithin for use in a method of enhancing T-cell fitness.

ix) A urolithin for use in a method of enhancing antigen presentation.

x) A urolithin for use as claimed in i) wherein the enhancement comprises upregulation of MHC Class I molecules.

xi) A urolithin for use as claimed in j) wherein the MHC Class I molecules are unregulated on epithelia cells.

xii) A urolithin for use as claimed in j) wherein the MHC Class I molecules are upregulated on tumour epithelia cells.

xiii) A urolithin for use in a method for the expansion of T-cells.

xiv) A urolithin for use as claimed in m) wherein the T-cells are T stem cells.

xv) A urolithin for use as claimed in m) wherein the T-cells are T memory stem cells.

xvi) A urolithin for use in a method of inducing CD8+ T cell immunity.

xvii) A urolithin for use in a method of enhancing CD8+ T cell dependent anti-tumour immunity.

xviii) A urolithin for use as defined in any one of a) to q) further comprising an immunotherapy treatment.

xix) A urolithin for use as claimed in any one of a) to q) 1 to 17 further comprising an immune checkpoint blockage therapy.

xx) A urolithin for use, as claimed in a) to s) wherein the use is for the treatment of diseases wherein there is T-cell dysfunction, for example, cancer and infectious diseases.

xxi) A urolithin for use, as claimed in t) wherein the cancer is selected from: bladder cancer melanoma, including paediatric melanoma, lung cancer, such as small cell lung cancer, non- small cell lung cancer, squamous cell lung carcinoma, head and neck cancer, such a head and neck squamous cell carcinoma, B-cell lymphoma, such as Hodgkin’s lymphoma, T-cell lymphoma, urothelial cancer, renal cancer, hepatocellular cancer, skin cancer, such as merkel cell carcinoma, gastric cancer and gastroesophageal cancer.

xxii) A urolithin for use as claimed in t) wherein the cancer is selected from: a microsatellite instability high (MSI-H) or mismatch repair deficient (dMMR) solid tumour.

xxiii) A urolithin for use as claimed in t) wherein the cancer is colorectal cancer.

xxiv) A urolithin for use as t) wherein the disease is an infectious disease.

xxv) A urolithin for use as claimed in x) wherein the infectious disease is selected from viral, bacterial and parasitic infections.

xxvi) A urolithin for use as claimed in s) wherein the immune checkpoint blockage therapy is selected from a PD-1 antagonist, an anti-CTLA4 therapy, an C28 antagonist, a B7-1 (CD80) and/or B7-2 (CD86) ligand antagonist, a CD27 antagonist, a CD40 antagonist, a CD40 Ligand, an OX40 antagonist, a GITR antagonist, a CD137 antagonist and/or a 41-BB-I antagonist.

xxvii) A urolithin for use as claimed in z) wherein the PD-1 antagonist is selected from an anti-PD-1 antibody, an anti-PD-L1 antibody or a fusion protein.

xxviii) A urolithin for use as claimed in a) to aa) further comprising one of more additional therapeutic agents.

xxix) A urolithin for use, as claimed in a) to bb) wherein the urolithin is urolithin A, urolithin B, urolithin C or urolithin D or a combination thereof.

xxx) A urolithin for use, as claimed in any one of a) to cc) wherein the urolithin is urolithin A.

### Statements of Invention (II)

(i) A urolithin for use in a method of enhancing antigen presentation.

(ii) A urolithin for use as claimed in claim 1 wherein the enhancement of antigen presentation comprises upregulation of MHC molecules.

(iii) A urolithin for use as claimed in claim 2 wherein the MHC molecules upregulation comprises upregulation of MHC class 1 molecules.

(iv) A urolithin for use as claimed in claim 2 or claim 3 wherein the MHC molecules are upregulated on epithelia cells, for example, tumour epithelia cells.

(v) A urolithin for use in a method of T-cell stem cell expansion, for example, T-memory stem cell expansion.

(vi) A urolithin for use in a method of inhibition of T stem cell differentiation, for example, T memory stem cell differentiation.

(vii) A urolithin for use as claimed in claim 5 or claim 6 wherein the urolithin is used substantially in the absence of nicotinamide riboside, manganese salts, for example manganese chloride and vitamin B12.

(viii) A urolithin for use, as claimed in any one of claims 1 to 4, for a method of treating a disease or condition where there is T-cell dysfunction.

(ix) A urolithin for use, as claimed in any one of claims 5 to 7, for a method of treating a disease of condition, where there is T-cell dysfunction.

(x) A urolithin for use, as claimed in claim 9 wherein the method comprises adoptive T-cell transfer.

(xi) A urolithin for use, as claimed in any one of claims 8 to 10, wherein the disease or condition is selected from cancer or an infectious disease.

(xii) A urolithin for use, as claimed in claim 8 to claim 11 wherein the treatment comprising enhancing CD8+ T cell dependent immunity.

(xiii) A urolithin for use in a method of overcoming or reversing of T-cell dysfunction, wherein the urolithin is used substantially in the absence of nicotinamide riboside, manganese salts, for example manganese chloride and vitamin B12..

(xiv) A urolithin for use as claimed in claim 14 wherein the T-cell dysfunction is T-cell exhaustion.

(xv) A urolithin for use as claimed in claim 14 wherein the T-cell dysfunction is T-cell exclusion.

(xvi) A urolithin for use as claimed in claim 15 wherein the T-cell exhaustion is CD8+ T-cell exhaustion

(xvii) A urolithin for use as claimed in claim 13 wherein the T-cell dysfunction is anergy.

(xviii) A urolthin for use as claimed in claim 17 wherein the anergy is CD8+ T-cell anergy.

(xix) A urolithin for use as claimed in claim 13 wherein the T-cell dysfunction is T-cell senescence.

(xx) A urolithin for use in a method of enhancing T-cell fitness, wherein the urolithin is used substantially in the absence of nicotinamide riboside, manganese salts, for example manganese chloride and vitamin B12.

(xxi) A urolithin for use, as claimed in claim 11 wherein the cancer is selected from: bladder cancer melanoma, including paediatric melanoma, lung cancer, such as small cell lung cancer, non- small cell lung cancer, squamous cell lung carcinoma, head and neck cancer, such a head and neck squamous cell carcinoma, B-cell lymphoma, such as Hodgkin’s lymphoma, T-cell lymphoma, urothelial cancer, renal cancer, hepatocellular cancer, skin cancer, such as merkel cell carcinoma, gastric cancer and gastroesophageal cancer, for example, colorectal cancer.

(xxii) A urolithin for use as claimed in claim 21 wherein the cancer is selected from: a microsatellite instability high (MSI-H) or mismatch repair deficient (dMMR) solid tumour.

(xxiii) A urolithin for use as claimed 11 wherein the disease is an infectious disease, selected from viral, bacterial and parasitic infections.

(xxiv) A urolithin for use as claimed in any one of the preceding claims further comprising one of more additional therapeutic agents.

(xxv) A urolithin for use, as claimed in any one of the preceding claims wherein the urolithin is urolithin A.

### Statements of Invention (III)

i) A method of preparing a T-cell population, enriched in T memory stem cells comprising administering a urolithin to a sample of T-cells.

ii) A method as claimed in claim 1, wherein the T cells, are transfected with a chimeric antigen receptor (CAR) gene.

iii) A method of preparing a T memory stem cell enriched CAR-T cell population comprising administering a urolithin to a population of CAR gene transfected T cells.

iv) A method of producing a population of urolithin-treated CAR-T cells, comprising:
a. Obtaining T cells from a subject;
b. Transfecting the T cells with a CAR (chimeric antigen receptor) gene, to prepare CAT-T cells; and
c. Administering a urolithin to the CAR-T cells, to produce a population of urolithin-treated CAR-T cells

v) A method as claimed in any one of claims 1 to 4 wherein the urolithin is urolithin A.

vi) A T-cell population obtained by a method as claimed in any one of clams 1 to 5.

vii) A T-cell population obtainable by a method as claimed in any one of clams 1 to 5.

viii) A T-cell population, enriched in T memory stem cells, obtained by a method as claimed in any one of clams 1 to 5.

ix) A T-cell population, enriched in T memory stem cells, obtainable by a method as claimed in any one of clams 1 to 5.

x) A T-cell population as claimed in any one of claims 6 to 9 for use in the treatment of a disease, for example, cancer.

xi) A T-cell population as claimed in any one of claims 6 to 9 for use in an adoptive cell therapy method for treating a disease, for example, cancer.

xii) CAR-T cells for use in an adoptive cell therapy method for treating cancer, comprising the step of administering CAR-T cells to a subject suffering from cancer, wherein the CAR-T cells have been pre-treated with a urolithin, for example, urolithin A.

1. A T-cell population enriched in T-memory stem cells.

2. The T-cell population of claim 1 wherein the population has been treated with a urolithin, for example, urolithin A.

3. The cell population claim 2 wherein the urolithin is administered at a concentration of between 10 µM and 100 µM, for example between 20 µM and 60 µM.

4. The T cell population of claim 1, wherein the T-memory stem cells comprise CD8 positive T-memory stem cells.

5. (canceled)

6. The T-cell population of claim 1, wherein the cells are transfected with a chimeric antigen receptor (CAR) gene.

7. A method of treating a disease, disorder or condition, comprising administering to a subject in need thereof an effective amount of the T-cell population of claim 2.

8. The method of claim 7, wherein the disease, disorder or condition is cancer, an autoimmune disease or an infectious disease.

9. The method of claim 8, wherein the cancer is selected from: bladder cancer melanoma, including paediatric melanoma, lung cancer, such as small cell lung cancer, non- small cell lung cancer, squamous cell lung carcinoma, head and neck cancer, such as head and neck squamous cell carcinoma, B-cell lymphoma, such as Hodgkin’s lymphoma, T-cell lymphoma, urothelial cancer, renal cancer, hepatocellular cancer, skin cancer, such as merkel cell carcinoma, gastric cancer and gastroesophageal cancer, for example, colorectal cancer.

10. A method of adoptive T cell transfer therapy, comprising administering to a subject in need thereof an effective amount of the T-cell population of claim 2.

11. The method of claim 10, wherein the disease, disorder or condition comprises T-cell dysfunction or T-cell exhaustion.

12. (canceled)

13. A method of preparing a T-cell population of claim 2, enriched in T memory stem cells, comprising administering a urolithin to a sample of T-cells.

14. The method of claim 13, wherein the T cells, are transfected with a chimeric antigen receptor (CAR) gene.

15. A method of preparing a T memory stem cell enriched CAR-T cell population comprising administering a urolithin to a population of CAR gene transfected T cells.

16. A method of producing a population of urolithin-treated CAR-T cells, comprising:
a) Obtaining T cells from a subject;
b) Transfecting the T cells with a CAR (chimeric antigen receptor) gene, to prepare CAR-T cells; and
c) Administering a urolithin to the CAR-T cells, to produce a population of urolithin-treated CAR-T cells.

17. (canceled)

18. T-cell population obtained by the method of claims 13, for example, a T-cell population, enriched in T memory stem cells.

19. (canceled)

20. A method of treatment of a disease, disorder or condition, for example, cancer, comprising administering to a subject in need thereof an effective amount of the T-cell population of claim 18.

21. A method of adoptive T-cell therapy for treating a disease, disorder or condition, for example, cancer, comprising administering to a subject in need thereof an effective amount of the T-cell population of claim 18.

22. A method of adoptive T-cell therapy for treating a disease, disorder of condition, for example, cancer, comprising the step of administering to a subject in need thereof an effective amount of CAR-T cells, wherein the CAR-T cells have been pre-treated with a urolithin, for example, urolithin A.

23. A a method of treating a disease, disorder or condition, for example, cancer or an infectious disease, comprising:
a) Isolating T cells from a patient in need thereof;
b) Culturing said T-cells ex-vivo; in the presence of a urolithin; and
c) Administering to the patient an effective amount of the urolithin-treated cells to the patient.

24. The method of claim 21, further comprising administering an immune checkpoint blockage therapy.
